(19) United States
(12) Patent Application Publication
(10) Pub. No.: US 2025/0332153 A1
(43) Pub. Date: Oct. 30, 2025

(54) TRANSDERMAL ANTICOAGULANT COMPOSITIONS

(71) Applicant: INTEGURX THERAPEUTICS, LLC, Califon, NJ (US)

(72) Inventors: Gary SHANGOLD, Califon, NJ (US); Hock TAN, East Brunswick, NJ (US); Agis KYDONIEUS, Kendall Park, NJ (US)

(21) Appl. No.: 18/997,356

(22) PCT Filed: Jul. 21, 2023
(86) PCT No.: PCT/US2023/070712

§ 371 Date: Jan. 21, 2025

Related U.S. Application Data
(60) Provisional application No. 63/391,127, filed on Jul. 21, 2022.

(51) Int. Cl. A61K 31/4545; A61K 9/70; A61K 31/19; A61K 47/12; A61K 47/20; A61K 47/26

(52) U.S. Cl. CPC A61K 31/4545; A61K 9/7061; A61K 31/19; A61K 47/12; A61K 47/20; A61K 47/26

(57) ABSTRACT

The present disclosure provides pharmaceutical compositions comprising an anticoagulant and pyruvic acid. Also provided are dosage forms comprising the pharmaceutical composition and methods for treating a thromboembolic disorder in a patient in need thereof, reducing the reoccurrence of a deep vein thrombosis or a pulmonary embolism in a patient in need thereof, preventing a venous thromboembolism (VTE) in an acutely ill patient, reducing the risk of a major cardiovascular event in a patient with coronary artery disease in a patient in need thereof, method for reducing the risk of a major thrombotic vascular event in a patient with peripheral artery disease (PAD), treating VTE or reducing the risk of reoccurrence of VTE in a pediatric patient in need thereof, thromboprophylaxis in a patient with congenital heart disease after a Fontan procedure, treating atherosclerosis, myocardial infarct, pulmonary embolism or deep venous thrombosis in a patient in need thereof, or preventing one or more thromboembolic or hemorrhagic events in a patient with atrial fibrillation by comprising administering the pharmaceutical composition or transdermal patch described herein to the patient.

### CROSS-REFERENCE TO RELATED APPLICATIONS

This application claims priority to U.S. Provisional Patent Application No. 63/391,127, filed Jul. 21, 2022, the disclosure of which is incorporated by reference.

### TECHNICAL FIELD

The disclosure relates to anticoagulant compositions and methods of using the same.

### BACKGROUND

Pharmaceutical products delivering the coagulation Factor Xa inhibitors apixaban (Eliquis®), rivaroxaban (Xarelto®), and edoxaban (Savaysa®), are used to prevent or treat deep vein thrombosis in at-risk patients such as those with atrial fibrillation or undergoing major knee or hip surgery. All are administered orally once or twice daily, and therapy is often prolonged or chronic.

All of the aforementioned products produce pharmacokinetic profiles characteristic of orally administered drugs, namely peak and trough patterns of circulating drug levels. Given normal inter-individual variability, peak levels following dosing may induce excessive anticoagulation, while trough levels which precede each subsequent dose may be associated with subtherapeutic degrees of anticoagulation (i.e. propensity toward thrombosis), with the relative activity of the coagulation cascade varying throughout each day. Consequently, a small but inescapable percentage of patients given these agents will experience either bleeding (hemorrhagic) or ischemic (thrombotic) events while on therapy. While some of these events are of minor consequence, significant and even fatal bleeding events occur regularly. Given the extremely widespread use of these drugs, the absolute numbers of patients who experience untoward safety events attributable to this problem is substantial.

A challenge with this class of drugs is that they do not readily lend themselves to efficient transport across the skin.

Safer and more efficient methods for administering anticoagulants to patients are needed.

### SUMMARY

In some aspects, the disclosure provides pharmaceutical compositions comprising an anticoagulant and pyruvic acid.

In other aspects, the disclosure provides dosage forms comprising the pharmaceutical composition described herein. In certain embodiments, the dosage form is a transdermal patch. In other embodiments, the dosage form is a transdermal gel.

In further aspects, the disclosure provides methods for treating a thromboembolic disorder in a patient in need thereof, comprising administering the pharmaceutical composition or dosage form, e.g., transdermal patch, described herein to the patient.

In yet other aspects, the disclosure provides methods for preventing a deep vein thrombosis or a pulmonary embolism in a patient in need thereof, comprising administering the pharmaceutical composition or dosage form, e.g., transdermal patch, described herein to the patient.

In still further aspects, the disclosure provides methods for reducing the reoccurrence of a deep vein thrombosis or a pulmonary embolism in a patient in need thereof, comprising administering the pharmaceutical composition or dosage form, e.g., transdermal patch, described herein to the patient.

In other embodiments, the disclosure provides methods for preventing a venous thromboembolism (VTE) in an acutely ill patient, comprising administering the pharmaceutical composition or dosage form, e.g., transdermal patch, described herein to the patient.

In further embodiments, the disclosure provides methods for reducing the risk of a major cardiovascular event in a patient with coronary artery disease, comprising administering the pharmaceutical composition or dosage form, e.g., transdermal patch, described herein to the patient.

In still other embodiments, the disclosure provides methods for reducing the risk of a major thrombotic vascular event in a patient with peripheral artery disease (PAD), comprising administering the pharmaceutical composition or dosage form, e.g., transdermal patch, described herein to the patient.

In yet further embodiments, the disclosure provides methods for treating VTE or reducing the risk of reoccurrence of VTE in a pediatric patient in need thereof, comprising administering the pharmaceutical composition or dosage form, e.g., transdermal patch, described herein to the patient.

In other embodiments, the disclosure provides methods for thromboprophylaxis in a patient with congenital heart disease after a Fontan procedure, comprising administering the pharmaceutical composition or dosage form, e.g., transdermal patch, described herein to the patient.

In further embodiments, the disclosure provides methods for treating atherosclerosis, myocardial infarct, pulmonary embolism or deep venous thrombosis in a patient in need thereof, comprising administering the pharmaceutical composition or dosage form, e.g., transdermal patch, described herein to the patient.

In yet other embodiments, the disclosure provides methods of preventing one or more thromboembolic events in a patient with atrial fibrillation, comprising administering the pharmaceutical composition or dosage form, e.g., transdermal patch, described herein to the patient.

In still further embodiments, the disclosure provides methods for reducing the risk of reducing the risk of stroke or systemic embolism in a patient, comprising administering the pharmaceutical composition or dosage form, e.g., transdermal patch, described herein to the patient. In some embodiments, the patient has nonvalvular atrial fibrillation.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 is a graph showing cumulative human skin permeation (μg/cm²) of apixaban utilizing formulations containing pyruvic acid. FIG. 1B is a graph showing the permeation rate (μg/cm²/h) of apixaban utilizing formulations containing pyruvic acid.

FIG. 2 is a graph showing cumulative human skin permeation (μg/cm²) of apixaban utilizing formulations containing pyruvic acid and the nonionic surfactant BRIJ O20. FIG. 2B is a graph showing the permeation rate (μg/cm²/h) of apixaban utilizing formulations containing pyruvic acid and the nonionic surfactant BRIJ O20.

FIG. 3 is a schematic of a transdermal reservoir patch for delivery of apixaban.

FIG. 4 is a graph showing cumulative human skin permeation (mg/cm²) of apixaban utilizing reservoir patches containing increasing concentrations of pyruvic acid.

FIG. 5 is a graph comparing cumulative human skin permeation (mg/cm²) of apixaban utilizing a reservoir patch or adhesive matrix patch.

FIG. 6 is drawing for a matrix transdermal patch.

FIG. 7 is a graph comparing the adhesive matrix patches of Example 5.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claimed invention.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

In the present disclosure the singular forms “a,” “an,” and “the” include the plural reference, and reference to a particular numerical value includes at least that particular value, unless the context clearly indicates otherwise. Thus, for example, a reference to “a therapeutic agent” is a reference to at least one of such therapeutic agents and equivalents thereof known to those skilled in the art, and so forth.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value.

When ranges are used herein for physical properties, such as amounts or weight %, all combinations, and subcombinations of ranges for specific embodiments therein are intended to be included.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The terms “patient” or “subject” as used herein refer to a mammalian animal and are used interchangeably. In some embodiments, the patient or subject is a human. In further embodiments, the patient or subject is an adult, i.e., ≥18 years of age. In yet other embodiments, the patient or subject is a child (a pediatric patient), i.e., <18 years of age. In still further embodiments, the patient or subject is ≥2 years of age. In other embodiments, the patient has atrial fibrillation such as nonvalvular atrial fibrillation. In further embodiments, the patient had hip or knee replacement surgery. In yet other embodiments, the patient has congenital heart disease after a Fontan procedure. In still further embodiments, the patient had recent lower extremity revascularization due to symptomatic peripheral artery disease (PAD). In yet other embodiments, the patient is acutely ill. In still further embodiments, the patient has coronary artery disease. In other embodiments, the patient has PAD.

“Treating” any disease or disorder refers, in some embodiments, to ameliorating a disease or disorder (i.e., arresting or reducing the development of the disease or at least one of the clinical symptoms thereof). In some embodiments, “treating” or “treatment” refers to ameliorating at least one physical parameter, which may not be discernible by the subject. In other embodiments, “treating” or “treatment” refers to modulating the disease or disorder, either physically, (e.g., stabilization of a discernible symptom), physiologically, (e.g., stabilization of a physical parameter), or both. In further embodiments, “treating” or “treatment” refers to delaying the onset of the disease or disorder.

The disclosure provides pharmaceutical compositions and dosage units for delivering anticoagulants to patients in need thereof. The compositions described herein permit permeation of anticoagulants through human skin. Desirably, the pharmaceutical compositions and dosage forms described herein lack acrylic acid, which deters permeation of the anticoagulant across human skin.

### Pharmaceutical Compositions

The present disclosure provides pharmaceutical compositions comprising an anticoagulant and pyruvic acid. The term “anticoagulant” as used herein refers to a chemical compound or small molecule that retards or inhibits coagulation of blood. In some embodiments, the anticoagulant is a thrombin inhibitor, Factor Xa inhibitor, a Factor XIa inhibitor, or a low molecular weight heparin. In other embodiments, the anticoagulant is a thrombin inhibitor. Examples of thrombin inhibitors include, without limitation, dabigatran, bivalirudin, lepirudin, or desirudin. In certain aspects, the thrombin inhibitor is dabigatran. In other embodiments, the anticoagulant is a low molecular weight heparin. Examples of low molecular weight heparins include, without limitation, enoxaparin. In further embodiments, the anticoagulant is a Factor Xa inhibitor. Examples of Factor Xa inhibitors include, without limitation, apixaban, rivaroxaban, edoxaban, betrixaban, or fondaparinux. In some aspects, the anticoagulant is apixaban. In yet other embodiments, the anticoagulant is a Factor XIa inhibitor. Examples of Factor XIa inhibitors include, without limitation, milvexian or asundexian.

The pharmaceutical compositions of the disclosure may contain about 5 to about 25% by weight, based on the weight of the pharmaceutical composition, of the anticoagulant. In some embodiments, the pharmaceutical compositions contains about 5, about 6, about 7, about 8, about 9, about 10, about 11, about 12, about 13, about 14, about 15, about 16, about 17, about 18, about 19, about 20, about 21, about 22, about 23, about 24, or about 25% by weight, based on the weight of the pharmaceutical composition, of the anticoagulant. In other embodiments, the pharmaceutical composition contains about 5 to about 24, about 5 to about 23, about 5 to about 22, about 5 to about 21, about 5 to about 20, 5 to about 19, about 5 to about 18, about 5 to about 17, about 5 to about 16, about 5 to about 15, about 5 to about 14, about 5 to about 13, about 5 to about 12, about 5 to about 11, about 5 to about 10, about 5 to about 9, about 5 to about 8, about 5 to about 7, about 5 to about 6, about 6 to about 25, about 6 to about 24, about 6 to about 23, about 6 to about 22, about 6 to about 21, about 6 to about 20, about 6 to about 19, about 6 to about 18, about 6 to about 17, about 6 to about 16, about 6 to about 15, about 6 to about 14, about 6 to about 13, about 6 to about 12, about 6 to about 11, about 6 to about 10, about 6 to about 9, about 6 to about 8, about 6 to about 7, about 7 to about 25, about 7 to about 24, about 7 to about 23, about 7 to about 22, about 7 to about 21, about 7 to about 20, about 7 to about 19, about 7 to about 18, about 7 to about 17, about 7 to about 16, about 7 to about 15, about 7 to about 14, about 7 to about 13, about 7 to about 12, about 7 to about 11, about 7 to about 10, about 7 to about 9, about 7 to about 8, about 8 to about 25, about 8 to about 24, about 8 to about 23, about 8 to about 22, about 8 to about 21, about 8 to about 20, about 8 to about 19, about 8 to about 18, about 8 to about 17, about 8 to about 16, about 8 to about 15, about 8 to about 14, about 8 to about 13, about 8 to about 12, about 8 to about 11, about 8 to about 10, about 8 to about 9, about 9 to about 25, about 9 to about 24, about 9 to about 23, about 9 to about 22, about 9 to about 21, about 9 to about 20, about 9 to about 19, about 9 to about 18, about 9 to about 17, about 9 to about 16, about 9 to about 15, about 9 to about 14, about 9 to about 13, about 9 to about 12, about 9 to about 11, about 9 to about 10, about 10 to about 25, about 10 to about 24,m about 10 to about 23, about 10 to about 22, about 10 to about 21, about 10 to about 20, about 10 to about 19, about 10 to about 18, about 10 to about 17, about 10 to about 16, about 10 to about 15, about 10 to about 14, about 10 to about 13, about 10 to about 12, about 10 to about 11, about 11 to about 25, about 11 to about 24, about 11 to about 23, about 11 to about 22, about 11 to about 21, about 11 to about 20, about 11 to about 19, about 11 to about 18, about 11 to about 17, about 11 to about 16, about 11 to about 15, about 11 to about 14, about 11 to about 13, about 11 to about 12, about 12 to about 25, about 12 to about 24, about 12 to about 23, about 12 to about 22, about 12 to about 21, about 12 to about 20, about 12 to about 19, about 12 to about 18, about 12 to about 17, about 12 to about 16, about 12 to about 15, about 12 to about 14, about 12 to about 13, about 13 to about 25, about 13 to about 24, about 13 to about 23, about 13 to about 22, about 13 to about 21 m, about 13 to about 20, about 13 to about 19, about 13 to about 18, about 13 to about 17, about 13 to about 16, about 13 to about 15, about 13 to about 14, about 14 to about 25, about 14 to about 24, about 14 to about 23, about 14 to about 22, about 14 to about 21, about 14 to about 20, about 14 to about 19, about 14 to about 18, about 14 to about 17, about 14 to about 16, about 14 to about 15, about 15 to about 25, about 15 to about 24, about 15 to about 23, about 15 to about 22, about 15 to about 21, about 15 to about 20, about 15 to about 19, about 15 to about 18, about 15 to about 17, about 15 to about 16, about 16 to about 25, about 16 to about 24, about 16 to about 23, about 16 to about 22, about 16 to about 21, about 16 to about 20, about 16 to about 19, about 16 to about 18, about 16 to about 17, about 17 to about 25, about 17 to about 24, about 17 to about 23, about 17 to about 22, about 17 to about 21, about 17 to about 20, about 17 to about 19, about 17 to about 18, about 18 to about 25, about 18 to about 24, about 18 to about 23, about 18 to about 22, about 18 to about 21, about 18 to about 20, about 18 to about 19, about 19 to about 25, about 19 to about 24, about 19 to about 23, about 19 to about 22, about 19 to about 21, about 19 to about 20, about 20 to about 25, about 20 to about 24, about 20 to about 23, about 20 to about 22, about 20 to about 21, about 21 to about 25, about 21 to about 24, about 21 to about 23, about 21 to about 22, about 22 to about 25, about 22 to about 24, about 22 to about 23, about 23 to about 25, about 23 to about 24 or about 24 to about 25% by weight, based on the weight of the pharmaceutical composition, of the anticoagulant. In further embodiments, the pharmaceutical composition contains about 7 to about 12% by weight, based on the weight of the pharmaceutical composition, of the anticoagulant. In yet other embodiments, the pharmaceutical composition contains about 9% by weight, based on the weight of the pharmaceutical composition, of the anticoagulant. In still further embodiments, the pharmaceutical composition contains about 10% by weight, based on the weight of the pharmaceutical composition, of the anticoagulant. In other embodiments, the pharmaceutical composition contains about 11% by weight, based on the weight of the pharmaceutical composition, of the anticoagulant.

According to the disclosure, the pharmaceutical composition also contains pyruvic acid. The pharmaceutical composition may contain about 20 to about 90% by weight, based on the weight of the pharmaceutical composition, of pyruvic acid. In some embodiments, the pharmaceutical composition contains about 20, about 25, about 30, about 35, about 40, about 45, about 50, about 55, about 60, about 65, about 70, about 75, about 80, about 85, or about 90% by weight, based on the weight of the pharmaceutical composition, of pyruvic acid. In other embodiments, the pharmaceutical composition contains about 20 to about 85, about 20 to about 80, about 20 to about 75, about 20 to about 70, about 20 to about 60, about 20 to about 55, about 20 to about 50, about 20 to about 45, about 20 to about 40, about 20 to about 35, about 20 to about 30, about 20 to about 25, about 25 to about 90, about 25 to about 85, about 25 to about 80, about 25 to about 75, about 25 to about 70, about 25 to about 65, about 25 to about 60, about 25 to about 55, about 25 to about 50, about 25 to about 45, about 25 to about 40, about 25 to about 35, about 25 to about 30, about 30 to about 90, about 30 to about 85, about 30 to about 80, about 30 to about 75, about 30 to about 70, about 30 to about 65, about 30 to about 60, about 30 to about 55, about 30 to about 50, about 30 to about 45, about 30 to about 40, about 30 to about 35, about 35 to about 90, about 35 to about 85, about 35 to about 80, about 35 to about 75, about 35 to about 70, about 35 to about 65, about 35 to about 60, about 35 to about 55, about 35 to about 50, about 35 to about 45, about 30 to about 40, about 40 to about 90, about 40 to about 85, about 40 to about 80, about 40 to about 75, about 40 to about 70, about 40 to about 65, about 40 to about 60, about 40 to about 55, about 40 to about 50, about 40 to about 45, about 45 to about 90, about 45 to about 85, about 45 to about 80, about 45 to about 75, about 45 to about 70, about 45 to about 65, about 45 to about 60, about 45 to about 55, about 45 to about 50, about 50 to about 90, about 50 to about 85, about 50 to about 80, about 50 to about 75, about 50 to about 70, about 50 to about 65, about 50 to about 60, about 50 to about 55, about 55 to about 90, about 55 to about 85, about 55 to about 80, about 55 to about 75, about 55 to about 70, about 55 to about 65, about 55 to about 60, about 60 to about 90, about 60 to about 85, about 60 to about 80, about 60 to about 75, about 60 to about 70, about 60 to about 65, about 65 to about 90, about 65 to about 85, about 65 to about 80, about 65 to about 75, about 65 to about 70, about 70 to about 90, about 70 to about 85, about 70 to about 80, about 70 to about 75, about 75 to about 90, about 75 to about 85, about 75 to about 80, about 80 to about 90, about 80 to about 85, or about 85 to about 90% by weight, based on the weight of the pharmaceutical composition, of pyruvic acid. In further embodiments, the pharmaceutical composition contains about 25 to about 45% by weight, based on the weight of the pharmaceutical composition, of pyruvic acid. In yet other embodiments, the pharmaceutical composition contains about 30 to about 45% by weight, based on the weight of the pharmaceutical composition, of pyruvic acid.

The pharmaceutical compositions described herein may also contain a surfactant, permeation enhancer, or a combination thereof. In some embodiments, the pharmaceutical composition contains a surfactant. In other embodiments, the pharmaceutical composition contain a permeation enhancer. In further embodiments, the pharmaceutical composition contains a surfactant and permeation enhancer. Examples of the surfactant or permeation enhancer include, without limitation, dimethylsulfoxide (DMSO); a low molecular weight acid such as 3-hydroxypropionic acid, lactic acid, oleic acid, levulinic acid, glycolic acid, or malonic acid; a nonionic surfactant; a polyethylene glycol monooleyl ether; dimethylacetamide (DMAc); or dimethyl isosorbide (DMI).

In certain aspects, the surfactant or permeation enhancer is dimethylsulfoxide (DMSO). The pharmaceutical composition may contain about 5 to about 20% by weight, based on the weight of the pharmaceutical composition, of DMSO. In some embodiments, the pharmaceutical composition contains about 5, about 6, about 7, about 8, about 9, about 10, about 11, about 12, about 13, about 14, about 15, about 16, about 17, about 18, about 19, or about 20% by weight, based on the weight of the pharmaceutical composition, of DMSO. In other embodiments, the pharmaceutical composition contains about 5 to about 19, about 5 to about 18, about 5 to about 17, about 5 to about 16, about 5 to about 15, about 5 to about 14, about 5 to about 13, about 5 to about 12, about 5 to about 11, about 5 to about 10, about 5 to about 9, about 5 to about 8, about 5 to about 7, about 5 to about 6, about 6 to about 20, about 6 to about 19, about 6 to about 18, about 6 to about 17, about 6 to about 16, about 6 to about 15, about 6 to about 14, about 6 to about 13, about 6 to about 12, about 6 to about 11, about 6 to about 10, about 6 to about 9, about 6 to about 8, about 6 to about 7, about 7 to about 20, about 7 to about 19, about 7 to about 18, about 7 to about 17, about 7 to about 16, about 7 to about 15, about 7 to about 14, about 7 to about 13, about 7 to about 12, about 7 to about 11, about 7 to about 10, about 7 to about 9, about 7 to about 8, about 8 to about 20, about 8 to about 19, about 8 to about 18, about 8 to about 17, about 8 to about 16, about 8 to about 15, about 8 to about 14, about 8 to about 13, about 8 to about 12, about 8 to about 11 m, about 8 to about 10, about 8 to about 9, about 9 to about 20, about 9 to about 19, about 9 to about 18, about 9 to about 17, about 9 to about 16, about 9 to about 15, about 9 to about 14, about 9 to about 13, about 9 to about 12, about 9 to about 11, about 9 to about 10, about 10 to about 20, about 10 to about 19,m about 10 to about 18, about 10 to about 17, about 10 to about 16, about 10 to about 15, about 10 to about 14, about 10 to about 13, about 10 to about 12, about 10 to about 11, about 11 to about 20, about 11 to about 19, about 11 to about 18, about 11 to about 17, about 11 to about 16, about 11 to about 15, about 11 to about 14, about 11 to about 13, about 11 to about 12, about 12 to about 20, about 12 to about 19, about 12 to about 18, about 12 to about 17, about 12 to about 16, about 12 to about 15, about 12 to about 14, about 12 to about 13, about 13 to about 20, about 13 to about 19, about 13 to about 18, about 13 to about 17, about 13 to about 16, about 13 to about 15, about 13 to about 14, about 14 to about 20, about 14 to about 19, about 14 to about 18, about 14 to about 17, about 14 to about 16, about 14 to about 15, about 15 to about 20, about 15 to about 19, about 15 to about 18, about 15 to about 17, about 15 to about 16, about 16 to about 20, about 16 to about 19, about 16 to about 18, about 16 to about 17, about 17 to about 20, about 17 to about 19, about 17 to about 18, about 18 to about 20, about 18 to about 19, or about 19 to about 20% by weight, based on the weight of the pharmaceutical composition, of DMSO. In further embodiments, the pharmaceutical composition contains about 8 to about 15% by weight, based on the weight of the pharmaceutical composition, of DMSO.

In other aspects, the surfactant or permeation enhancer is lactic acid. The pharmaceutical composition may contain about 20 to about 45% by weight, based on the weight of the pharmaceutical composition, of lactic acid. In some embodiment, the pharmaceutical composition contains about 20, about 21, about 22, about 23, about 24, about 25, about 26, about 27 about 28, about 29, about 30, about 31, about 32, about 33, about 34, about 35, about 36, about 37, about 38, about 39, about 40, about 41, about 42, about 43, about 44, or about 45% by weight, based on the weight of the pharmaceutical composition, of lactic acid. In other embodiments, the pharmaceutical composition contains about 20 to about 40, about 20 to about 35, about 20 to about 30, about 20 to about 25, about 25 to about 45, about 25 to about 40, about 25 to about 35, about 25 to about 30, about 30 to about 45, about 30 to about 40, about 30 to about 35, about 35 to about 45, about 30 to about 40, or about 40 to about 45% by weight, based on the weight of the pharmaceutical composition, of lactic acid. In further embodiments, the pharmaceutical composition contains about 25 to about 35% by weight, based on the weight of the pharmaceutical composition, of lactic acid.

In further aspects, the surfactant or permeation enhancer is oleic acid. The pharmaceutical composition may contain about 1 to about 10% by weight, based on the weight of the pharmaceutical composition, of oleic acid. In some embodiments, the pharmaceutical composition contains about 1, about 2, about 3, about 4, about 5, about 6, about 7, about 8, about 9 or about 10% by weight, based on the weight of the pharmaceutical composition, of lactic acid. In other embodiments, the pharmaceutical composition contains about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2 to about 3, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10% by weight, based on the weight of the pharmaceutical composition, of lactic acid. In further embodiments, the pharmaceutical composition contains about 3 to about 7% by weight, based on the weight of the pharmaceutical composition, of lactic acid. In yet other embodiments, the pharmaceutical compositions contains about 1 to about 5% by weight, based on the weight of the pharmaceutical compositions, of lactic acid.

In yet other aspects, the surfactant or permeation enhancer is levulinic acid. The pharmaceutical compositions may contain about 1 to about 20% by weight, based on the weight of the pharmaceutical composition, of levulinic acid. In some embodiments, the pharmaceutical compositions contain about 1, about 2, about 3, about 4, about 5, about 6, about 7, about 8, about 9, about 10, about 11, about 12, about 13, about 14, about 15, about 16, about 17, about 18, about 19, or about 20. In other embodiments, the pharmaceutical compositions contain about 1 to about 18, about 1 to about 16, about 1 to about 14, about 1 to about 12, about 1 to about 10, about 1 to about 8, about 1 to about 6, about 1 to about 4, about 1 to about 3, about 3 to about 20, about 3 to about 18, about 3 to about 16, about 3 to about 14, about 3 to about 12, about 3 to about 10, about 3 to about 8, about 3 to about 6, about 3 to about 5, about 5 to about 19, about 5 to about 18, about 5 to about 17, about 5 to about 16, about 5 to about 15, about 5 to about 14, about 5 to about 13, about 5 to about 12, about 5 to about 11, about 5 to about 10, about 5 to about 9, about 5 to about 8, about 5 to about 7, about 5 to about 6, about 6 to about 20, about 6 to about 19, about 6 to about 18, about 6 to about 17, about 6 to about 16, about 6 to about 15, about 6 to about 14, about 6 to about 13, about 6 to about 12, about 6 to about 11, about 6 to about 10, about 6 to about 9, about 6 to about 8, about 6 to about 7, about 7 to about 20, about 7 to about 19, about 7 to about 18, about 7 to about 17, about 7 to about 16, about 7 to about 15, about 7 to about 14, about 7 to about 13, about 7 to about 12, about 7 to about 11, about 7 to about 10, about 7 to about 9, about 7 to about 8, about 8 to about 20, about 8 to about 19, about 8 to about 18, about 8 to about 17, about 8 to about 16, about 8 to about 15, about 8 to about 14, about 8 to about 13, about 8 to about 12, about 8 to about 11, about 8 to about 10, about 8 to about 9, about 9 to about 20, about 9 to about 19, about 9 to about 18, about 9 to about 17, about 9 to about 16, about 9 to about 15, about 9 to about 14, about 9 to about 13, about 9 to about 12, about 9 to about 11, about 9 to about 10, about 10 to about 20, about 10 to about 19,m about 10 to about 18, about 10 to about 17, about 10 to about 16, about 10 to about 15, about 10 to about 14, about 10 to about 13, about 10 to about 12, about 10 to about 11, about 11 to about 20, about 11 to about 19, about 11 to about 18, about 11 to about 17, about 11 to about 16, about 11 to about 15, about 11 to about 14, about 11 to about 13, about 11 to about 12, about 12 to about 20, about 12 to about 19, about 12 to about 18, about 12 to about 17, about 12 to about 16, about 12 to about 15, about 12 to about 14, about 12 to about 13, about 13 to about 20, about 13 to about 19, about 13 to about 18, about 13 to about 17, about 13 to about 16, about 13 to about 15, about 13 to about 14, about 14 to about 20, about 14 to about 19, about 14 to about 18, about 14 to about 17, about 14 to about 16, about 14 to about 15, about 15 to about 20, about 15 to about 19, about 15 to about 18, about 15 to about 17, about 15 to about 16, about 16 to about 20, about 16 to about 19, about 16 to about 18, about 16 to about 17, about 17 to about 20, about 17 to about 19, about 17 to about 18, about 18 to about 20, about 18 to about 19, or about 19 to about 20% by weight, based on the weight of the pharmaceutical composition, of levulinic acid. In further embodiments, the pharmaceutical compositions contain about 3 to about 10% by weight, based on the weight of the pharmaceutical composition, of levulinic acid.

In still further aspects, the surfactant or permeation enhancer is 3-hydroxypropionic acid. In some embodiments, the pharmaceutical composition contains about 20, about 25, about 30, about 35, about 40, about 45, about 50, about 55, about 60, about 65, about 70, about 75, about 80, about 85, or about 90% by weight, based on the weight of the pharmaceutical composition, of 3-hydroxypropionic acid. In other embodiments, the pharmaceutical composition contains about 20 to about 85, about 20 to about 80, about 20 to about 75, about 20 to about 70, about 20 to about 60, about 20 to about 55, about 20 to about 50, about 20 to about 45, about 20 to about 40, about 20 to about 35, about 20 to about 30, about 20 to about 25, about 25 to about 90, about 25 to about 85, about 25 to about 80, about 25 to about 75, about 25 to about 70, about 25 to about 65, about 25 to about 60, about 25 to about 55, about 25 to about 50, about 25 to about 45, about 25 to about 40, about 25 to about 35, about 25 to about 30, about 30 to about 90, about 30 to about 85, about 30 to about 80, about 30 to about 75, about 30 to about 70, about 30 to about 65, about 30 to about 60, about 30 to about 55, about 30 to about 50, about 30 to about 45, about 30 to about 40, about 30 to about 35, about 35 to about 90, about 35 to about 85, about 35 to about 80, about 35 to about 75, about 35 to about 70, about 35 to about 65, about 35 to about 60, about 35 to about 55, about 35 to about 50, about 35 to about 45, about 30 to about 40, about 40 to about 90, about 40 to about 85, about 40 to about 80, about 40 to about 75, about 40 to about 70, about 40 to about 65, about 40 to about 60, about 40 to about 55, about 40 to about 50, about 40 to about 45, about 45 to about 90, about 45 to about 85, about 45 to about 80, about 45 to about 75, about 45 to about 70, about 45 to about 65, about 45 to about 60, about 45 to about 55, about 45 to about 50, about 50 to about 90, about 50 to about 85, about 50 to about 80, about 50 to about 75, about 50 to about 70, about 50 to about 65, about 50 to about 60, about 50 to about 55, about 55 to about 90, about 55 to about 85, about 55 to about 80, about 55 to about 75, about 55 to about 70, about 55 to about 65, about 55 to about 60, about 60 to about 90, about 60 to about 85, about 60 to about 80, about 60 to about 75, about 60 to about 70, about 60 to about 65, about 65 to about 90, about 65 to about 85, about 65 to about 80, about 65 to about 75, about 65 to about 70, about 70 to about 90, about 70 to about 85, about 70 to about 80, about 70 to about 75, about 75 to about 90, about 75 to about 85, about 75 to about 80, about 80 to about 90, about 80 to about 85, or about 85 to about 90% by weight, based on the weight of the pharmaceutical composition, of 3-hydroxypropionic acid. In further embodiments, the pharmaceutical composition contains about 25 to about 45% by weight, based on the weight of the pharmaceutical composition, of 3-hydroxypropionic acid. In yet other embodiments, the pharmaceutical composition contains about 30 to about 45% by weight, based on the weight of the pharmaceutical composition, of 3-hydroxypropionic acid.

In other aspects, the permeation enhancer is malonic acid. In some embodiments, the pharmaceutical composition contains about 20, about 25, about 30, about 35, about 40, about 45, about 50, about 55, about 60, about 65, about 70, about 75, about 80, about 85, or about 90% by weight, based on the weight of the pharmaceutical composition, of malonic acid. In other embodiments, the pharmaceutical composition contains about 20 to about 85, about 20 to about 80, about 20 to about 75, about 20 to about 70, about 20 to about 60, about 20 to about 55, about 20 to about 50, about 20 to about 45, about 20 to about 40, about 20 to about 35, about 20 to about 30, about 20 to about 25, about 25 to about 90, about 25 to about 85, about 25 to about 80, about 25 to about 75, about 25 to about 70, about 25 to about 65, about 25 to about 60, about 25 to about 55, about 25 to about 50, about 25 to about 45, about 25 to about 40, about 25 to about 35, about 25 to about 30, about 30 to about 90, about 30 to about 85, about 30 to about 80, about 30 to about 75, about 30 to about 70, about 30 to about 65, about 30 to about 60, about 30 to about 55, about 30 to about 50, about 30 to about 45, about 30 to about 40, about 30 to about 35, about 35 to about 90, about 35 to about 85, about 35 to about 80, about 35 to about 75, about 35 to about 70, about 35 to about 65, about 35 to about 60, about 35 to about 55, about 35 to about 50, about 35 to about 45, about 30 to about 40, about 40 to about 90, about 40 to about 85, about 40 to about 80, about 40 to about 75, about 40 to about 70, about 40 to about 65, about 40 to about 60, about 40 to about 55, about 40 to about 50, about 40 to about 45, about 45 to about 90, about 45 to about 85, about 45 to about 80, about 45 to about 75, about 45 to about 70, about 45 to about 65, about 45 to about 60, about 45 to about 55, about 45 to about 50, about 50 to about 90, about 50 to about 85, about 50 to about 80, about 50 to about 75, about 50 to about 70, about 50 to about 65, about 50 to about 60, about 50 to about 55, about 55 to about 90, about 55 to about 85, about 55 to about 80, about 55 to about 75, about 55 to about 70, about 55 to about 65, about 55 to about 60, about 60 to about 90, about 60 to about 85, about 60 to about 80, about 60 to about 75, about 60 to about 70, about 60 to about 65, about 65 to about 90, about 65 to about 85, about 65 to about 80, about 65 to about 75, about 65 to about 70, about 70 to about 90, about 70 to about 85, about 70 to about 80, about 70 to about 75, about 75 to about 90, about 75 to about 85, about 75 to about 80, about 80 to about 90, about 80 to about 85, or about 85 to about 90% by weight, based on the weight of the pharmaceutical composition, of malonic acid. In further embodiments, the pharmaceutical composition contains about 25 to about 45% by weight, based on the weight of the pharmaceutical composition, of malonic acid. In yet other embodiments, the pharmaceutical composition contains about 30 to about 45% by weight, based on the weight of the pharmaceutical composition, of malonic acid.

In further aspects, the surfactant or permeation enhancer is a nonionic surfactant. Examples of nonionic enhancers include, without limitation, polysorbates such as polysorbates 80, e.g., Tween® 80. The pharmaceutical composition may contain about 5 to about 15% by weight, based on the weight of the pharmaceutical composition, of the nonionic enhancer. In some embodiments, the pharmaceutical composition contains about 5, about 6, about 7, about 8, about 9, about 10, about 11, about 12, about 13, about 14, or about 15% by weight, based on the weight of the pharmaceutical composition, of the nonionic enhancer. In other embodiments, the pharmaceutical composition contains about 5 to about 14, about 5 to about 13, about 5 to about 12, about 5 to about 11, about 5 to about 10, about 5 to about 9, about 5 to about 8, about 5 to about 7, about 5 to about 6, about 6 to about 15, about 6 to about 14, about 6 to about 13, about 6 to about 12, about 6 to about 11, about 6 to about 10, about 6 to about 9, about 6 to about 8, about 6 to about 7, about 7 to about 15, about 7 to about 14, about 7 to about 13, about 7 to about 12, about 7 to about 11, about 7 to about 10, about 7 to about 9, about 7 to about 8, about 8 to about 14, about 8 to about 13, about 8 to about 12, about 8 to about 11, about 8 to about 10, about 8 to about 9, about 9 to about 14, about 9 to about 13, about 9 to about 12, about 9 to about 11, about 9 to about 10, about 10 to about 15, about 10 to about 14, about 10 to about 13, about 10 to about 12, about 10 to about 11, about 11 to about 15, about 11 to about 14, about 11 to about 13, about 11 to about 12, about 12 to about 15, about 12 to about 14, about 12 to about 13, about 13 to about 15, about 13 to about 14, or about 14 to about 15% by weight, based on the weight of the pharmaceutical composition, of the nonionic enhancer. In further embodiments, the pharmaceutical compositions contains about 7 to about 12% by weight, based on the weight of the pharmaceutical composition, of the nonionic enhancer. In yet other embodiments, the pharmaceutical composition contains about 0.5 to about 2% by weight, based on the weight of the pharmaceutical composition, of the nonionic enhancer.

In yet other aspects, the surfactant or permeation enhancer is a polyethylene glycol monooleyl ether. Examples of polyethylene glycol monooleyl ethers include polyoxyethylene (20) oleyl ether, e.g., Brij® O20. The pharmaceutical composition may contain about 0.01 to about 5% by weight, based on the weight of the pharmaceutical composition, of the polyethylene glycol monooleyl ether. In some embodiments, the pharmaceutical composition contains about 0.5, about 1, about 1.5, about 2, about 2.5, about 3, about 3.5, about 4, about 4.5 or about 5% by weight, based on the weight of the pharmaceutical composition, of the polyethylene glycol monooleyl ether. In other embodiments, the pharmaceutical composition contains about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 5, about 1 to about 4.5 m about 1 to about 4, about 1 to about 3.5, about 1 to about 3, about 1 to about 2.5, about 1 to about 2, about 1 to about 1.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 5, about 2 to about 4.5, about 2 to about 4, about 2 to about 3.5, about 2 to about 3, about 2 to about 2.5, about 2.5 to about 5, about 2.5 to about 4.5, about 2.5 to about 4, about 2.5 to about 3.5, about 2.5 to about 3, about 3 to about 5, about 3 to about 4.5, about 3 to about 4, about 3 to about 3.5, about 3.5 to about 5, about 3.5 to about 4.5, about 3.5 to about 4, about 4 to about 5, about 4 to about 4.5 or about 4.5 to about 5% by weight, based on the weight of the pharmaceutical composition, of the polyethylene glycol monooleyl ether. In further embodiments, the pharmaceutical composition contains about 0.5 to about 2% by weight, based on the weight of the pharmaceutical composition, of the polyethylene glycol monooleyl ether.

The pharmaceutical compositions of the disclosure may be designed to be administered to the skin or mucosal tissue of a patient in need of treatment. In addition to the anticoagulant and pyruvic acid, the pharmaceutical compositions of the disclosure may also include excipients. Examples of suitable excipients include, without limitation, further comprises one or more of a filler, binder, disintegrant, emulsifier, anti-static agent, or solvent.

### Dosage Forms

The present disclosure also contemplates dosage forms comprising the pharmaceutical compositions described herein. Thus, the pharmaceutical compositions may be formulated as gels, transdermal patches, lotions, creams, sprays, mists, emulsions, or dispersions. Appropriate excipients for formulating a gel, transdermal patch, lotion, cream, spray, or mist are readily apparent to a person of skill in the art and include, but are not limited to, stabilizers, emulsifiers, thickeners, antimicrobials, humectants, propellants, spreading agents, polymers, and adhesives, such as pressure sensitive adhesives. In particular, excipients that may be used to form a transdermal gel include, but are not limited to, alcohols, glycols, glycerin, butylated hydroxytoluene (BHT), and water. In some embodiments, the dosage form is a transdermal patch. In other embodiments, the dosage form is a gel. In further embodiments, the dosage form is a lotion. In yet other embodiments, the dosage form is a cream. In still further embodiments, the dosage form is a spray. In other embodiments, the dosage form is a mist. In further embodiments, the dosage form is an emulsion. In still other embodiments, the dosage form is a dispersion.

The dosage form may be designed for delivery of the pharmaceutical composition over a period of time that is dependent on the patient. In some embodiments, the dosage form is designed for delivery of the anticoagulant over a period of one day, i.e., 24 hours. In other embodiments, the dosage form is designed for delivery of the anticoagulant over a period of 2 days, e.g., 48 hours. In further embodiments, the dosage form is designed for delivery of the anticoagulant over a period of 3 days. In still further embodiments, the dosage form is designed for delivery of the anticoagulant over a period of 3.5 days. In yet other embodiments, the dosage form is designed for delivery of the anticoagulant over a period of 4 days. In still further embodiments, the dosage form is designed for delivery of the anticoagulant over a period of 5 days. In other embodiments, the dosage form is designed for delivery of the anticoagulant over a period of 6 days. In further embodiments, the dosage form is designed for delivery of the anticoagulant over a period of 7 days, e.g., 1 week.

The particular form of the transdermal patch may be selected by one skilled in the art. In some embodiments, the transdermal patch is a single-layer drug-in-adhesive patch, multi-layer drug-in-adhesive patch, reservoir patch, matrix patch, or a vapor patch. In other embodiments, the dosage form is a single-layer drug-in-adhesive patch. In further embodiments, the dosage form is a multi-layer drug-in-adhesive patch. In yet other embodiments, the dosage form is a reservoir patch. In still further embodiments, the dosage form is a matrix patch. In other embodiments, the dosage form is a vapor patch.

The amount of anticoagulant present in the dosage form depends on the daily dosage of anticoagulant to be administered and on the particular anticoagulant selected. Thus, if the dosage form is intended to release the anticoagulant for more than 1 day, the amount of anticoagulant is adjusted accordingly. In some embodiments, the dosage form administers the anticoagulant over a period of 2 days and the amount of anticoagulant is 2× the daily dose. In some embodiments, the dosage form administers the anticoagulant over a period of 3 days and the amount of anticoagulant is 3× the daily dose. In some embodiments, the dosage form administers the anticoagulant over a period of 4 days and the amount of anticoagulant is 4× the daily dose. In some embodiments, the dosage form administers the anticoagulant over a period of 5 days and the amount of anticoagulant is 5× the daily dose. In some embodiments, the dosage form administers the anticoagulant over a period of 6 days and the amount of anticoagulant is 6× the daily dose. In some embodiments, the dosage form administers the anticoagulant over a period of 7 days, i.e., 1 week, and the amount of anticoagulant is 7× the daily dose.

In some embodiments, the dosage form contains an excess of the anticoagulant to ensure that a concentration gradient that favors flux from the patch across the skin throughout the period of wear is maintained. The term “excess” as used herein refers to an amount that is about 2 to about 5× more than the weight of the anticoagulant in the dosage form. In some embodiments, an excess is an about 2-fold excess, i.e., about 2× the weight of the anticoagulant in the dosage form. In other embodiments, an excess is an about 3-fold excess, i.e., about 3× the weight of the anticoagulant in the dosage form. In further embodiments, an excess is an about 4-fold excess, i.e., about 4× the weight of the anticoagulant in the dosage form. In still other embodiments, an excess is an about 5-fold excess, i.e., about 5× the weight of the anticoagulant in the dosage form. By way of example, the following formula may be used to calculate the amount of anticoagulant in the dosage form:

$$Amount of anticoagulant in the dosage form ( mg ) = ( daily dosage of anticoagulant ( mg ) ) \times ( \# days of dosage form water ) \times ( fold excess of anticoagulant in the dosage form )$$

For example, if the daily dosage of the anticoagulant is about 100 mg and the dosage unit is designed to release the anticoagulant over a period of seven days, then the dosage form contains about 1400 mg (100 mg×2-fold excess×7 days) to about 3500 mg (100 mg×5-fold excess×7 days).

In some embodiments, the dosage form contains an amount of apixaban that delivers about 2.5 to about 20 mg/day of apixaban to the patient. In certain aspects, the dosage form contains an amount of apixaban that delivers about 2.5, about 3, about 4, about 5, about 6, about 7, about 8, about 9, about 10, about 11, about 12, about 13, about 14, about 15, about 16, about 17, about 18, about 19 or about 20 mg/day of apixaban to the patient. In other aspects, the dosage form contains an amount of apixaban that delivers about 2.5 to about 17.5, about 2.5 to about 15, about 2.5 to about 12.5, about 2.5 to about 10, about 2.5 to about 7.5, about 7.5 to about 5, about 5 to about 20, about 5 to about 17.5, about 5 to about 15, about 5 to about 12.5, about 5 to about 10, about 5 to about 7.5, about 7.5 to about 20, about 7.5 to about 17.5, about 7.5 to about 15, about 7.5 to about 12.5, about 7.5 to about 10, about 10 to about 20, about 10 to about 17.5, about 10 to about 15, about 10 to about 12.5, about 12.5 to about 20, about 12.5 to about 17.5, about 12.5 to about 15, about 15 about 20, about 15 to about 17.5 or about 17.5 to about 20 mg/day of apixaban to the patient.

In some embodiments, the transdermal dosage forms described herein deliver the same amount of apixaban in about 24 hours as oral dosage forms comprising apixaban. For example, in some aspects, the transdermal dosage forms will exhibit the same or similar (i.e., about 80% to about 125%) area under the curve (AUC) as oral dosage forms comprising apixaban. In other embodiments, the transdermal dosage forms described herein result in a Cₘₐₓ that is not the same or similar to the Cₘₐₓ achieved using oral dosage forms comprising apixaban. In some aspects, the transdermal dosage forms will exhibit a Cₘₐₓ that is lower, e.g., about 10%, about 15%, about 20%, about 25%, or about 30% lower, than the Cₘₐₓ exhibited when an oral dosage form comprising apixaban is administered. In further embodiments, the transdermal dosage forms described herein described herein result in a trough level that is above the trough level achieved using oral dosage forms of apixaban. Without wishing to be bound to any particular theory, transdermal dosage forms described herein will be therapeutically equivalent to oral dosage forms containing apixaban and the described transdermal dosage forms will have a comparable or improved safety profile, as compared to oral dosage forms comprising apixaban.

In other embodiments, the dosage form contains an amount of rivaroxaban that delivers about 2.5 to about 30 mg/day of rivaroxaban to the patient. In certain aspects, the dosage form contains an amount of rivaroxaban that delivers about 2.5, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/day of rivaroxaban to the patient. In other aspects, the dosage form contains an amount of rivaroxaban that delivers about 2.5 to about 30, about 2.5 to about 27.5, about 2.5 to about 25, about 2.5 to about 22.5, about 2.5 to about 20, about 2.5 to about 17.5, about 2.5 to about 15, about 2.5 to about 12.5, about 2.5 to about 10, about 2.5 to about 7.5, about 2.5 to about 5, about 5 to about 30, about 5 to about 27.5, about 5 to about 25, about 5 to about 22.5, about 5 to about 20, about 5 to about 17.5, about 5 to about 15, about 5 to about 12.5, about 5 to about 10, about 5 to about 7.5, about 7.5 to about 30, about 7.5 to about 27.5, about 7.5 to about 25, about 7.5 to about 22.5, about 7.5 to about 20, about 7.5 to about 17.5, about 7.5 to about 15, about 7.5 to about 12.5, about 7.5 to about 10, about 10 to about 30, about 10 to about 27.5, about 10 to about 25, about 10 to about 22.5, about 10 to about 20, about 10 to about 17.5, about 10 to about 15, about 10 to about 12.5, about 12.5 to about 30, about 12.5 to about 27.5, about 12.5 to about 25, about 12.5 to about 22.5, about 12.5 to about 20, about 12.5 to about 17.5, about 12.5 to about 15, about 15 to about 30, about 15 to about 27.5, about 15 to about 25, about 15 to about 22.5, about 15 about 20, about 15 to about 17.5, about 17.5 to about 30, about 17.5 to about 27.5, about 17.5 to about 25, about 17.5 to about 22.5, about 17.5 to about 20, about 20 to about 30, about 20 to about 27.5, about 20 to about 25, about 20 to about 22.5, about 22.5 to about 30, about 22.5 to about 27.5, about 22.5 to about 25, about 25 to about 30, about 25 to about 27.5, or about 27.5 to about 30 mg/day of rivaroxaban to the patient.

In further embodiments, the dosage form contains an amount of edoxaban that delivers about 15 to about 60 mg/day of edoxaban to the patient. In certain aspects, the dosage form contains an amount of edoxaban that delivers about 15, about 20, about 25, about 30, about 35, about 40, about 45, about 50, about 55, or about 60 mg/day of edoxaban to the patient. In further aspects, the dosage form contains an amount of edoxaban that delivers about 15 to about 55, about 15 to about 50, about 15 to about 45, about 15 to about 40, about 15 to about 35, about 15 to about 30, about 15 to about 25, about 15 to about 20, about 20 to about 60, about 20 to about 55, about 20 to about 50, about 20 to about 45, about 20 to about 40, about 20 to about 35, about 20 to about 30, about 20 to about 25, about 25 to about 60, about 25 to about 55, about 25 to about 50, about 25 to about 45, about 25 to about 40, about 25 to about 35, about 25 to about 30, about 30 to about 60, about 30 to about 55, about 30 to about 50, about 30 to about 45, about 30 to about 40, about 30 to about 35, about 35 to about 60, about 35 to about 55, about 35 to about 50, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60 mg/day of edoxaban to the patient. In other aspects, the dosage form contains an amount of edoxaban that delivers about 15 to about 30 mg/day of edoxaban to the patient.

In yet other embodiments, the dosage form contains an amount of betrixaban that delivers about 40 to about 160 mg/day of betrixaban to the patient. In certain aspects, the dosage form contains an amount of betrixaban that delivers about 40, about 50, about 60, about 70, about 80, about 90, about 100, about 110, about 120, about 130, about 140, about 150, or about 160 mg/day of betrixaban to the patient. In further aspects, the dosage form contains an amount of betrixaban that delivers about 40 to about 140, about 40 to about 120, about 40 to about 100, about 40 to about 80, about 40 to about 60, about 60 to about 160, about 60 to about 140, about 60 to about 120, about 60 to about 100, about 60 to about 80, about 80 to about 160, about 80 to about 140, about 80 to about 120, about 80 to about 100, about 100 to about 160, about 100 to about 140,m about 100 to about 120, about 120 to about 160, about 120 to about 140, or about 140 to about 160 mg/day of betrixaban to the patient. In other embodiments, the dosage form contains an amount of betrixaban that delivers about 80 to about 160 mg/day of betrixaban to the patient.

In still further embodiments, the dosage form contains an amount of fondaparinux that delivers about 2.5 to about 10 mg/day of fondaparinux to the patient. In certain aspects, the dosage form contains an amount of fondaparinux that delivers about 2.5, about 3, about 4, about 5, about 6, about 7, about 8, about 9, or about 10 mg/day of fondaparinux to the patient. In other aspects, the dosage form contains an amount of fondaparinux that delivers about 2.5 to about 10, about 2.5 to about 7.5, about 7.5 to about 5, about 5 to about 10, about 5 to about 7.5, or about 7.5 to about 10 mg/day of fondaparinux to the patient.

In other embodiments, the dosage form contains an amount of dabigatran that delivers about 75 to about 220 mg of dabigatran to the patient. In certain aspects, the dosage form contains an amount of dabigatran that delivers about 75, about 100, about 125, about 150, about 175, about 200, or about 220 mg of dabigatran to the patient. In other aspects, the dosage form contains an amount of dabigatran that delivers about 75 to about 200, about 75 to about 175, about 75 to about 150, about 75 to about 125, about 75 to about 100, about 100 to about 220, about 100 to about 200, about 100 to about 175, about 100 to about 150, about 100 to about 125, about 125 to about 220, about 125 to about 200, about 125 to about 175, about 125 to about 150, about 150 to about 220, about 150 to about 200, about 150 to about 175, about 175 to about 220, about 175 to about 200, or about 200 to about 220 mg/day of dabigatran to the patient. In further aspects, the dosage form contains an amount of dabigatran that delivers about 75 to about 150 mg/day of dabigatran to the patient.

In further embodiments, the dosage form contains an amount of enoxaparin that delivers about 30 to about 60 mg/day of enoxaparin to the patient. In certain aspects, the dosage form contains an amount of enoxaparin that delivers about 30, about 35, about 40, about 45, about 50, about 55, or about 60 mg/day of enoxaparin to the patient. In further aspects, the dosage form contains an amount of enoxaparin that delivers about 30 to about 60, about 30 to about 55, about 30 to about 50, about 30 to about 45, about 30 to about 40, about 30 to about 35, about 35 to about 60, about 35 to about 55, about 35 to about 50, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60 mg/day of enoxaparin to the patient. In other aspects, the dosage form contains an amount of enoxaparin that delivers about 40 mg/day of enoxaparin to the patient.

In yet other embodiments, the dosage form contains an amount of milvexian that delivers about 25 to about 500 mg/day of milvexian to the patient. In certain aspects, the dosage form contains an amount of milvexian that delivers about 25, about 50, about 60, about 100, about 150, about 200, about 250, about 300, about 350, about 400, about 450, or about 500 mg/day of milvexian to the patient. In further aspects, the dosage form contains an amount of milvexian that delivers about 25 to about 450, about 25 to about 400, about 25 to about 350, about 25 to about 300, about 25 to about 250, about 25 to about 200, about 25 to about 150, about 25 to about 100, about 25 to about 60, about 25 to about 50, about 50 to about 500, about 50 to about 450, about 50 to about 400, about 50 to about 350, about 50 to about 300, about 50 to about 250, about 50 to about 200, about 50 to about 150, about 50 to about 100, about 50 to about 60, about 60 to about 500, about 60 to about 450, about 60 to about 400, about 60 to about 350, about 60 toa bout 300, about 60 to about 250, about 60 to about 200, about 60 to about 150, about 60 to about 100, about 100 to about 500, about 100 to about 450, about 100 to about 400, about 100 to about 350, about 100 to about 300, about 100 to about 250, about 100 to about 200, about 100 to about 150, about 150 to about 500, about 150 to about 450, about 150 to about 400, about 150 to about 350, about 150 to about 300, about 150 to about 250, about 150 to about 200, about 200 to about 500, about 200 to about 450, about 200 to about 400, about 200 to about 350, about 200 to about 300, about 200 to about 250, about 250 to about 500, about 250 to about 450, about 250 to about 400, about 250 to about 350, about 250 to about 300, about 300 to about 500, about 300 to about 450, about 300 to about 400, about 400 to about 350, about 350 to about 500, about 350 to about 450, about 350 to about 400, about 400 to about 500, about 400 to about 450, or about 450 to about 500 mg/day of milvexian to the patient. In yet other aspects, the dosage form contains an amount of milvexian that delivers about 50 to about 200 mg/day of milvexian to the patient. In still further aspects, the dosage form contains an amount of milvexian that delivers about 60 to about 200 mg/day of milvexian to the patient. In other aspects, the dosage form contains an amount of milvexian that delivers about 50 to about 500 mg/day of milvexian to the patient. In further aspects, the dosage form contains an amount of milvexian that delivers about 60 to about 500 mg/day of milvexian to the patient.

In still further embodiments, the dosage form contains an amount of asundexian that delivers about 10 to about 50 mg/day of asundexian to the patient. In certain aspects, the dosage form contains an amount of asundexian that delivers about 10, about 15, about 20, about 25, about 30, about 30, about 35, about 40, about 45, or about 50 mg/day of asundexian to the patient. In further aspects, the dosage form contains an amount of asundexian that delivers about 10 to about 50, about 10 to about 45, about 10 to about 40, about 10 to about 35, about 10 to about 30, about 10 to about 25, about 10 to about 20, about 10 to about 15, about 15 to about 50, about 15 to about 45, about 15 to about 40, about 15 to about 35, about 15 to about 30, about 15 to about 25, about 15 to about 20, about 20 to about 50, about 20 to about 45, about 20 to about 40, about 20 to about 35, about 20 to about 30, about 20 to about 25, about 25 to about 50, about 25 to about 45, about 25 to about 40, about 25 to about 35, about 25 to about 30, about 30 to about 50, about 30 to about 45, about 30 to about 40, about 30 to about 35, about 35 to about 50, about 35 to about 45, about 35 to about 40, about 40 to about 50, about 40 to about 45, or about 45 to about 50, mg/day of asundexian to the patient. In other aspects, the dosage form contains an amount of asundexian that delivers about 20 to about 50 mg/day of asundexian to the patient.

The dosage form may also contain a pressure sensitive adhesive that adheres the dosage form, e.g., transdermal patch, to the skin of the patient. In some embodiments, the pressure sensitive adhesive is an acrylate pressure sensitive adhesive, a silicon-based pressure sensitive adhesive, a polyisobutylene based pressure sensitive adhesive, a hybrid acrylate-silicone based pressure sensitive adhesive, or a combination thereof. See, e.g., Tan and Pfister, “Pressure-sensitive adhesives for transdermal drug delivery systems,” Pharm Sci & Tech Today 2:60-69 (1999), which is incorporated by reference. In other embodiments, the pressure sensitive adhesive contains a acrylate copolymer with OH groups and vinyl acetate. In further embodiments, the pressure sensitive adhesive is DUROTAK 87-2287 or DUROTAK 4098. The dosage form contains about 35 to about 95% by weight, based on the weight of the dosage form, of the pressure sensitive adhesive. In some embodiments, the dosage form contains about 35, about 40, about 45, about 50, about 55, about 60, about 65, about 70, about 75, about 80, about 85, about 90, or about 95%, based on the weight of the dosage form, of the pressure sensitive adhesive. In further embodiments, the dosage form contains about 35 to about 90, about 35 to about 85, about 35 to about 80, about 35 to about 75, about 35 to about 70, about 35 to about 65, about 35 to about 60, about 35 to about 55, about 35 to about 50, about 35 to about 45, about 40 to about 95, about 40 to about 90, about 40 to about 85, about 40 to about 80, about 40 to about 75, about 40 to about 70, about 40 to about 65, about 40 to about 60, about 40 to about 55, about 40 to about 50, about 50 to about 95, about 50 to about 90, about 50 to about 80, about 50 to about 75, about 50 to about 70, about 50 to about 65, about 50 to about 60, about 60 to about 95, about 60 to about 90, about 60 to about 85, about 60 to about 80, about 60 to about 75, about 60 to about 70, about 70 to about 95, about 70 to about 90, about 70 to about 85, about 70 to about 80, about 80 to about 95, or about 80 to about 90% by weight, based on the weight of the dosage form, of the pressure sensitive adhesive.

### Methods of Treatment

According to the disclosure, methods or uses of using the pharmaceutical compositions and/or dosage forms also are provided. Regardless of the particular method, the methods includes administering the pharmaceutical composition or dosage form to the patient. In some embodiments, the disclosure provides methods for treating a thromboembolic disorder in a patient in need thereof. The term “thromboembolic disorder” as used herein refers to a disorder where blood clots form in blood vessels. In some aspects, the thromboembolic disorder is an arterial cardiovascular thromboembolic disorder, a venous cardiovascular thromboembolic disorder, or a thromboembolic disorder in the chambers of the heart. In other aspects, the thromboembolic disorder is an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis (DVT), thrombophlebitis, arterial embolism, coronary arterial thrombosis, cerebral arterial thrombosis, cerebral embolism, kidney embolism, pulmonary embolism (PE), or a thrombosis resulting from or a procedure in which blood is exposed to an artificial surface that promotes thrombosis such as thrombosis resulting from an implant such as a prosthetic valve, thrombosis resulting from an indwelling catheter, thrombosis resulting from a stent, thrombosis resulting from a cardiopulmonary bypass, thrombosis resulting from hemodialysis, thrombosis resulting from or a procedure in which blood is exposed to an artificial surface that promotes thrombosis. In further aspects, the thromboembolic disorder is an acute coronary syndrome. In yet other aspects, the thromboembolic disorder is stroke. In still further aspects, the thromboembolic disorder is deep DVT. In other aspects, the thromboembolic disorder is pulmonary embolism (PE).

The disclosure also provides methods for treating, reducing the reoccurrence, or preventing a deep vein thrombosis (DVT) and/or a pulmonary embolism (PE) in a patient in need thereof. In some embodiments, the methods treat a DVT or PE. In other embodiments, the methods reduce the reoccurrence of a DVT and/or PE. In further embodiments, the methods prevent a DVT and/or PE. In certain aspects, the patient had hip or knee replacement surgery. In other aspects, the patient had hip replacement surgery. In further aspects, the patient had knee replacement surgery.

According to the disclosure, methods for treating atherosclerosis, myocardial infarct, pulmonary embolism (PE) or deep venous thrombosis (DVT) in a patient in need thereof are provided. The methods include administering the pharmaceutical composition or dosage form to the patient. In some embodiments, the methods treat atherosclerosis. In further embodiments, the methods treat myocardial infarct. In other embodiments, the methods treat pulmonary embolism. In yet further embodiments, the methods treat deep venous thrombosis.

According to the disclosure, methods for treating, reducing the risk of reoccurrence, or preventing a venous thromboembolism (VTE) are provided and include administering the pharmaceutical composition or dosage form described herein to the patient. In some embodiments, the disclosure provides methods for preventing a VTE. In other embodiments, the disclosure provides methods for treating a VTE. In further aspects, the disclosure provides methods for reducing the risk of reoccurrence of VTE. In certain aspects, the patient is acutely ill. In other aspects, the patient is a pediatric patient.

The disclosure further provides methods for reducing the risk of a major cardiovascular event in a patient. These methods include administering the pharmaceutical composition or dosage form to the patient. In certain aspects, the patient has coronary artery disease. In some aspects, the patient has acute myocardial infarction, i.e., heart attack. In other aspects, the patient has a pulmonary embolism. In further aspects, the patient has heart failure. In yet other aspects, the patient has a stroke.

According to the disclosure, methods for reducing the risk of a major thrombotic vascular event in a patient are provided. These methods include administering the pharmaceutical composition or dosage form to the patient. In certain aspects, the patient has peripheral artery disease (PAD). In other aspects, the patient had recent lower extremity revascularization due to symptomatic PAD. In further aspects, the patient has deep vein thrombosis. In yet other aspects, the patient has systemic embolus. In still further aspects, the patient has thrombophlebitis. In other aspects, the patient has a pulmonary embolus.

The disclosure also provides methods for thromboprophylaxis in a patient. The methods include administering the pharmaceutical composition or dosage form to the patient. In some aspects, the patient has congenital heart disease after a Fontan procedure. In other aspects, the patient is 2 years of age or older.

The disclosure further provides methods of preventing one or more thromboembolic events in a patient. The term “thromboembolic disorder” as used herein refers to a condition that leads to a hypercoagulable state of the blood, or a propensity for generating clots. In some embodiments, a thromboembolic disorder can lead to thromboembolic event. The term “thromboembolic event” as used herein refers to a condition wherein a blood clot (thrombus) is formed and optionally transported to another area of the body. The methods include administering the pharmaceutical composition or dosage form to the patient. In some embodiments, the patient has atrial fibrillation.

The disclosure also provides methods for reducing the risk of reducing the risk of stroke or systemic embolism in a patient. The methods include administering the pharmaceutical composition or dosage form to the patient. In some aspects, the patient has nonvalvular atrial fibrillation.

For all of the methods of the disclosure, the pharmaceutical composition or dosage form may be administered transdermally.

The methods also include administering more than one anticoagulant described herein. The additional or second anticoagulant may be selected by the attending physician. In some embodiments, the second anticoagulant is aspirin. In further embodiments, the second anticoagulant is clopidogrel.

### ASPECTS

Aspect 1: A pharmaceutical composition comprising an anticoagulant and pyruvic acid.

Aspect 2: The pharmaceutical composition of Aspect 1, wherein the anticoagulant is a thrombin inhibitor, Factor Xa inhibitor, a Factor Xia inhibitor, or a low molecular weight heparin.

Aspect 3: The pharmaceutical composition of Aspect 1, wherein the thrombin inhibitor is dabigatran, bivalirudin, lepirudin, or desirudin, or such as dabigatran.

Aspect 4: The pharmaceutical composition of Aspect 1, wherein the low molecular weight heparin is enoxaparin.

Aspect 5: The pharmaceutical composition of Aspect 1, wherein the Factor Xa inhibitor is apixaban, rivaroxaban, edoxaban, betrixaban, or fondaparinux, or a combination thereof, or such as apixaban.

Aspect 6: The pharmaceutical composition of Aspect 1, wherein the Factor XIa inhibitor is milvexian or asundexian.

Aspect 7: The pharmaceutical composition of any one of the preceding Aspects, further comprising a surfactant, permeation enhancer, or a combination thereof.

Aspect 8: The pharmaceutical composition of Aspect 7, wherein the surfactant or permeation enhancer is dimethylsulfoxide, lactic acid, oleic acid, levulinic acid, 3-hydroxypropionic acid, malonic acid, or a nonionic surfactant such as a polysorbate such as Tween 80, or a polyethylene glycol monooleyl ether, such as Brij O20.

Aspect 9: The pharmaceutical composition of any one of the preceding Aspects, further comprising lactic acid.

Aspect 10. The pharmaceutical composition of Aspect 8 or 9, comprising about 20 to about 45% by weight, based on the weight of the pharmaceutical composition, of lactic acid, or such as about 25 to about 35% by weight.

Aspect 11: The pharmaceutical composition of any one of the preceding Aspects, further comprising dimethylsulfoxide.

Aspect 12: The pharmaceutical composition of Aspect 11, comprising about 5 to about 20% by weight, based on the weight of the pharmaceutical composition, of dimethylsulfoxide, or about 8 to about 15% by weight.

Aspect 13: The pharmaceutical composition of any one of the preceding Aspects, further comprising levulinic acid.

Aspect 14: The pharmaceutical composition of Aspect 13, comprising about 1 to about 20% by weight, based on the weight of the pharmaceutical composition, of levulinic acid, ore such as about 3 to about 10% by weight.

Aspect 15: The pharmaceutical composition of any one of the preceding Aspects, further comprising oleic acid.

Aspect 16: The pharmaceutical composition of Aspect 15, comprising about 1 to about 10% by weight, based on the weight of the pharmaceutical composition, of oleic acid, or such as about 3 to about 7% by weight.

Aspect 17. The pharmaceutical composition of any one of the preceding Aspects, further comprising a polysorbate, or such as polysorbate 80.

Aspect 18: The pharmaceutical composition of Aspect 17, comprising about 5 to about 15% by weight, based on the weight of the pharmaceutical composition, of the polysorbate, or such as about 7 to about 12% by weight.

Aspect 19: The pharmaceutical composition of any one of the preceding Aspects, further comprising a polyethylene glycol monooleyl ether.

Aspect 20: The pharmaceutical composition of Aspect 19, comprising about 0.01 to about 5% by weight, based on the weight of the pharmaceutical composition, of the polyethylene glycol monooleyl ether, or such as about 0.5 to about 2% by weight.

Aspect 21: The pharmaceutical composition of any one of the preceding Aspects, comprising about 5 to about 25% by weight, based on the weight of the pharmaceutical composition, of the anticoagulant, or such as about 7 to about 12% by weight, such as about 9%, about 10%, or about 11% by weight.

Aspect 22: The pharmaceutical composition of any one of the preceding Aspects, comprising about 20 to about 90% by weight, based on the weight of the pharmaceutical composition, of pyruvic acid, or such as about 25 to about 45% by weight, or about 30 to about 45% by weight.

Aspect 23: The pharmaceutical composition of any one of the preceding Aspects, comprising about 0.1 to about 100 mg/day, or such as about 2.5 mg/day, about 5 mg/day, about 10 mg/day, about 15 mg/day, or about 20 mg of the anticoagulant.

Aspect 24: The pharmaceutical composition of any one of the preceding Aspects that lacks acrylic acid.

Aspect 25: A dosage form comprising the pharmaceutical composition of any one of the preceding Aspects.

Aspect 26: The dosage form of Aspect 25 that is a transdermal patch.

Aspect 27: The dosage form of Aspect 26, wherein the transdermal patch is a single-layer drug-in-adhesive patch, multi-layer drug-in-adhesive patch, reservoir patch, matrix patch, or a vapor patch, or such as a reservoir patch.

Aspect 28. A method for treating a thromboembolic disorder in a patient in need thereof, comprising administering the pharmaceutical composition of any one of Aspects 1-24 or the dosage form of any one of Aspects 25-27 to the patient.

Aspect 29: The method of Aspect 28, wherein the thromboembolic disorder is an arterial cardiovascular thromboembolic disorder, venous cardiovascular thromboembolic disorder, or thromboembolic disorder in the chambers of the heart.

Aspect 30: The method of Aspect 28, wherein the thromboembolic disorder is an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or a thrombosis resulting from or a procedure in which blood is exposed to an artificial surface that promotes thrombosis such as thrombosis resulting from an implant such as a prosthetic valve, thrombosis resulting from an indwelling catheter, thrombosis resulting from a stent, thrombosis resulting from a cardiopulmonary bypass, thrombosis resulting from hemodialysis, thrombosis resulting from or a procedure in which blood is exposed to an artificial surface that promotes thrombosis.

Aspect 31: The method of Aspect 28, wherein the thromboembolic disorder is an acute coronary syndrome.

Aspect 32: The method of Aspect 28, wherein the thromboembolic disorder is stroke.

Aspect 33: The method of Aspect 28, wherein the thromboembolic disorder is deep vein thrombosis.

Aspect 34: The method of Aspect 28, wherein the thromboembolic disorder is pulmonary embolism.

Aspect 35: A method for preventing a deep vein thrombosis or a pulmonary embolism in a patient in need thereof, comprising administering the pharmaceutical composition of any one of Aspects 1-24 or the dosage form of any one of Aspects 25-27 to the patient.

Aspect 36: A method for reducing the reoccurrence of a deep vein thrombosis or a pulmonary embolism in a patient in need thereof, comprising administering the pharmaceutical composition of any one of Aspects 1-24 or the dosage form of any one of Aspects 25-27 to the patient.

Aspect 37: The method of Aspect 35 or 36, wherein the patient had hip or knee replacement surgery.

Aspect 38: A method for preventing a venous thromboembolism (VTE) in an acutely ill patient, comprising administering the pharmaceutical composition of any one of claims 1-24 or the dosage form of any one of Aspects 25-27 to the patient.

Aspect 39: A method for reducing the risk of a major cardiovascular event in a patient with coronary artery disease, comprising administering the pharmaceutical composition of any one of Aspects 1-24 or the dosage form of any one of Aspects 25-27 to the patient.

Aspect 40: A method for reducing the risk of a major thrombotic vascular event in a patient with peripheral artery disease (PAD), comprising administering the pharmaceutical composition of any one of Aspects 1-24 or the dosage form of any one of claims 25-27 to the patient.

Aspect 41: The method of Aspect 40, wherein the patient had recent lower extremity revascularization due to symptomatic PAD.

Aspect 42: A method for treating VTE or reducing the risk of reoccurrence of VTE in a pediatric patient in need thereof, comprising administering the pharmaceutical composition of any one of Aspects 1-24 or the dosage form of any one of Aspects 25-27 to the patient.

Aspect 43: A method for thromboprophylaxis in a patient with congenital heart disease after a Fontan procedure, comprising administering the pharmaceutical composition of any one of Aspects 1-24 or the dosage form of any one of Aspects 25-27 to the patient.

Aspect 44: The method of Aspect 42, wherein the patient is 2 years of age or older.

Aspect 45: A method for treating atherosclerosis, myocardial infarct, pulmonary embolism or deep venous thrombosis in a patient in need thereof, comprising administering the pharmaceutical composition of any one of Aspects 1-24 or the dosage form of any one of Aspects 25-27 to the patient.

Aspect 46: The method of any one of Aspects 28-45, wherein the pharmaceutical composition is administered transdermally.

Aspect 47. The method of any one of Aspects 28-46, further comprising administering a second anticoagulant.

Aspect 48: The method of Aspect 47, wherein the second anticoagulant is aspirin.

Aspect 49: The method of any one of Aspects 35-41 or 43-48, wherein the patient is an adult.

Aspect 50: The method of any one of Aspects 35-49, wherein the patient is a child.

Aspect 51: A method of preventing one or more thromboembolic events in a patient with atrial fibrillation, comprising administering the pharmaceutical composition of any one of Aspects 1-24 or the dosage form of any one of Aspects 25-27 to the patient.

The following examples, while illustrative individual embodiments, are not intended to limit the scope of the described invention, and the reader should not interpret them in this way.

### EXAMPLES

### Example 1: Effect of pyruvic acid and BRIJ O20 on skin permeation

Apixaban formulations of Table 1 were prepared to study the effect of enhancers, solvents and emulsifiers on the permeation of apixaban through human cadaver skin. The formulations were mixed under vortex conditions until all the apixaban was dissolved. The mixing time was dependent on the size of the vessel, but in general the time was approximately 15 minutes. Formulation F6 was based on a formulation described in US Patent Publication No. 2020/0338012.

TABLE 1

| | Weight % | | |
|---|---|---|---|
| Component | F6 | F7 | F8 |
| DMSO | 0 | 12.8 | 9.4 |
| Pyruvic Acid | 0 | 36.4 | 35.7 |
| Lactic Acid | 40.1 | 31.9 | 26.8 |
| Acrylic acid | 40.1 | 0 | 0 |
| Levulinic Acid | 0 | 4.5 | 4.4 |
| Oleic Acid | 0 | 4.6 | 4.5 |
| TWEEN 80 | 8.9 | 0 | 9.0 |
| BRIJ O20 | 0 | 1.0 | 0 |
| Apixaban | 11.0 | 8.9 | 10.2 |

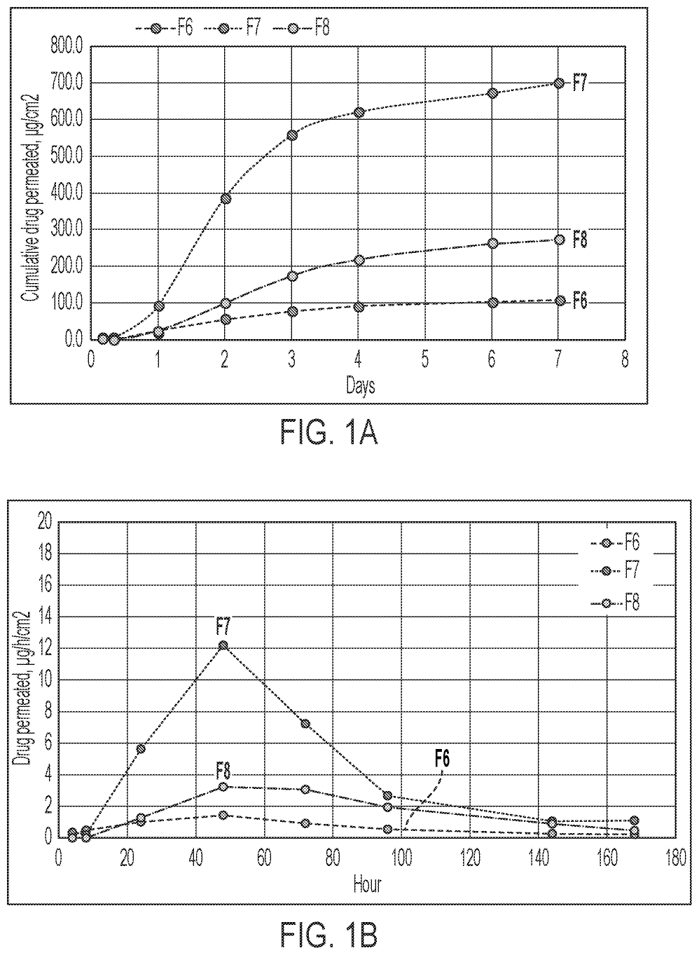

Skin permeation studies were performed for the formulations using Franz diffusion cells kept at 37° C. for the duration of the experiment. The receptor medium was phosphate buffered saline at pH 7.4, the receptor volume 12 mL and the permeation area 1.77 cm². Human cadaver skin was used, and all tests were performed in triplicate. Each of the formulations (0.4 mL) was placed at the donor site of the skin diffusion cells and the experiment was initiated with the receptor medium being continuously mixed. Samples of the receptor phase were obtained at 1, 2, 3, 4, 5 and 7 days, and apixaban concentrations were determined using HPLC method [Column: Agilent column C18, 3.5 μm, 4.6 mm×150 mm; Mobile Phase: water (0.025% formic acid):acetonitrile (50:50); Gradient method; Flow Rate: 1 mL/min; Detection; 280 nm; Column Temperature: 40° C.]. The skin flux for the three formulations was determined and is shown in FIGS. 1A and 1B.

FIG. 1 shows that the formulation that contained pyruvic acid and the nonionic surfactant BRIJ O20 (F7) gave the highest flux through human skin. The formulation that lacked pyruvic acid (F6) gave the lowest skin permeation, although it contained a nonionic surfactant, TWEEN 80, at a high level. The formulation that contained pyruvic acid and TWEEN 80 (F8) gave somewhat higher skin flux than formulation F6, but still substantially lower than formulation F7. Some conclusions that can be drawn from this experiment are a) pyruvic acid increases skin permeation, b) pyruvic acid together with the surfactant BRIJ O20 provides a synergistic effect on skin permeation, c) surfactants appear to be important for the delivery of apixaban, though many other aspects of the surfactant are also important, such as hydrophilic-lipophilic balance (HLB) and melting point (MP).

### Example 2: Effect of Pyruvic Acid and BRIJ O20 on Skin Permeation of AXB at Constant Amount of DMSO

To verify the importance of pyruvic acid and BRIJ O20 on the apixaban skin permeation, three formulations were prepared as shown in Table 2.

TABLE 2

| | Weight % | | |
|---|---|---|---|
| Component | F7 | F9 | F10 |
| DMSO | 12.8 | 12.9 | 12.7 |
| Pyruvic Acid | 36.4 | 36.7 | 24.2 |
| Lactic Acid | 31.9 | 32.2 | 32.4 |
| Oleic Acid | 4.5 | 4.5 | 4.5 |
| Levulinic Acid | 4.6 | 4.6 | 16.3 |
| BRIJ O20 | 1.0 | 0 | 1.0 |
| Apixaban | 8.9 | 9.0 | 8.9 |

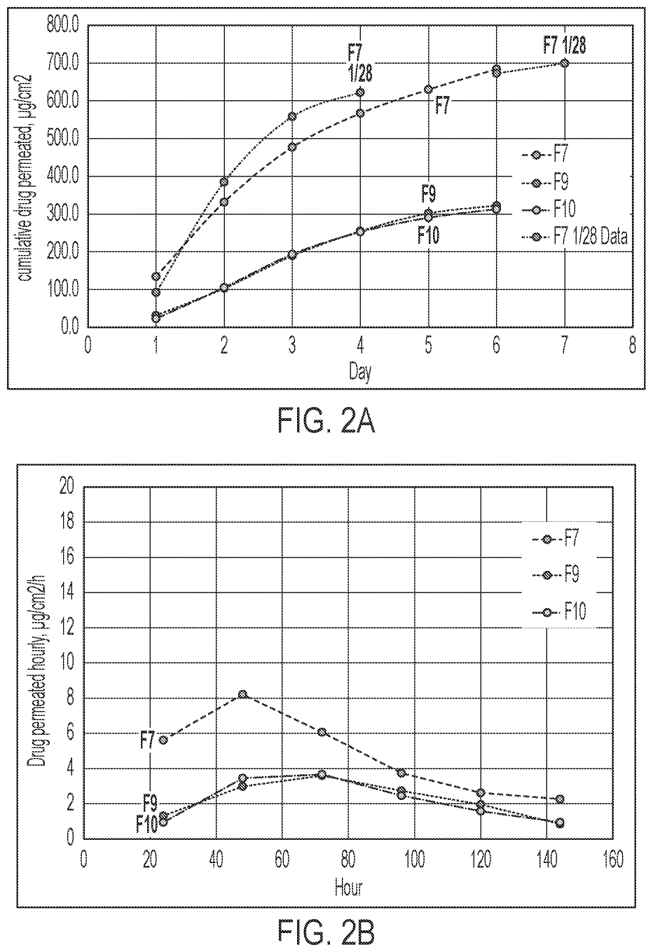

All three formulations were tested in Franz Diffusion cells, as described in Example 1, and skin permeation as a function of time was plotted as shown in FIGS. 2A and 2B. FIGS. 2A and 2B show the skin permeation of formulation F7 from Example 2 (marked as ‘ 1/28 data’). The F7 curves show similar skin flux, which is well within the expected error for skin flux experiments, thus demonstrating reproducibility of the results of that experiment.

The skin permeation data substantiates the conclusions reached in Example 2, i.e., pyruvic acid and BRIJ O20 are enhancers for the skin permeation of apixaban, and they are independent of influences by other excipients.

In formulation F10, the concentration of levulinic acid was increased by proportionately reducing the concentration of pyruvic acid, and the result was a marked reduction in apixaban skin flux. This indicates that the utility of the oleic acid/levulinic combination is of limited value in enhancing the transdermal delivery of apixaban, and that pyruvic acid is largely responsible for the increased flux discovered in these experiments.

### Example 3: Preparation and Testing of Reservoir Transdermal Patches

Reservoir transdermal patches were prepared containing the formulation F7 as the saturated solution within the patch. Formulation F7 was shown in Examples 2 and 3 to give the best skin permeation of any apixaban formulations tested. Considering that pyruvic acid was shown herein to be a good enhancer for apixaban, formulations F12 and F14, containing higher amounts of pyruvic acid, were prepared. The composition of the three formulations is given in Table 3.

TABLE 3

| | Weight % | | |
|---|---|---|---|
| Component | F7 | F12 | F14 |
| DMSO | 12.8 | 0 | 0 |
| Pyruvic Acid | 36.4 | 48.5 | 79.7 |
| Lactic Acid | 31.9 | 31.5 | 0 |
| Oleic Acid | 4.5 | 4.4 | 4.4 |
| Levulinic Acid | 4.6 | 4.5 | 4.5 |
| BRIJ O20 | 1.0 | 1.0 | 1.0 |
| Apixaban | 8.9 | 10.1 | 10.5 |
| Total | 100.0 | 100.0 | 100.0 |

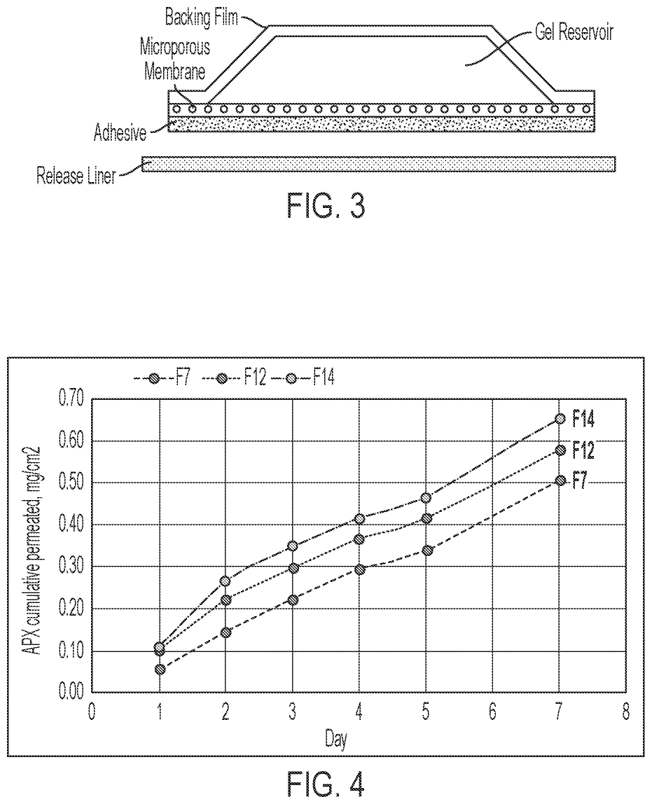

A schematic of the reservoir transdermal patches prepared is shown in FIG. 3. The patches are composed of four components: an impermeable backing, a drug reservoir which contains the drug dissolved in vehicle (i.e., active gel), a microporous membrane, and an adhesive. A polypropylene membrane at 38% porosity (CELGARD 2400) was selected as the rate-controlling membrane. A heat-sealable, polyester film laminate (SCOTCHPAK 1012, 3M) comprises the backing layer. Pressure sensitive adhesive (i.e., DUROTAK4098) is cast onto the release liner (SCOTCHPAK 9747, 3M), and dried. A layer of CELGARD is placed atop the pressure sensitive adhesive. A backing film is laid on top of the membrane, and heat sealed the rim of a 1.77 cm² circle using a die compressed for 10 seconds at 70° C. to form an empty pouch reservoir between the CELGARD and backing layer. The active gel formulation was loaded into the patch reservoir by inserting a hypodermic syringe into the orifice leading into the reservoir chamber, filling it with 0.4 mL of apixaban gel solution and then sealing the orifice.

The three reservoir patches prepared were placed on the human cadaver skin, and then mounted in the Franz cell as used above (1.77 cm² area) for the skin flux study. The skin permeation study was performed as described in Example 2. The amount of apixaban permeated in mg/cm² was determined and plotted versus time (7-day period) as shown in FIG. 4. The results showed that skin permeation was better with the higher amount of pyruvic acid in the patch.

### Example 4

Two formulations were prepared: a gel formulation suitable for use as a reservoir patch (formulation F15) and an adhesive matrix patch formulation using DUROTAK 87-2287 (Comparison formulation #1). The final compositions for the two formulations are provided in Table 4.

TABLE 4

| | Formulation F15 | Comparison #1 |
|---|---|---|
| Component | Dry weight % | dry weight % |
| Pyruvic Acid | 79.65 | 49.94% |
| Oleic Acid | 4.40 | 2.76% |
| Levulinic Acid | 4.52 | 2.83% |
| BRIJ O20 | 0.95 | 0.60% |
| Apixaban | 10.47 | 6.56% |
| DUROTAK 87-2287 | 0 | 37.30% |
| Total | 100.00 | 100.00 |

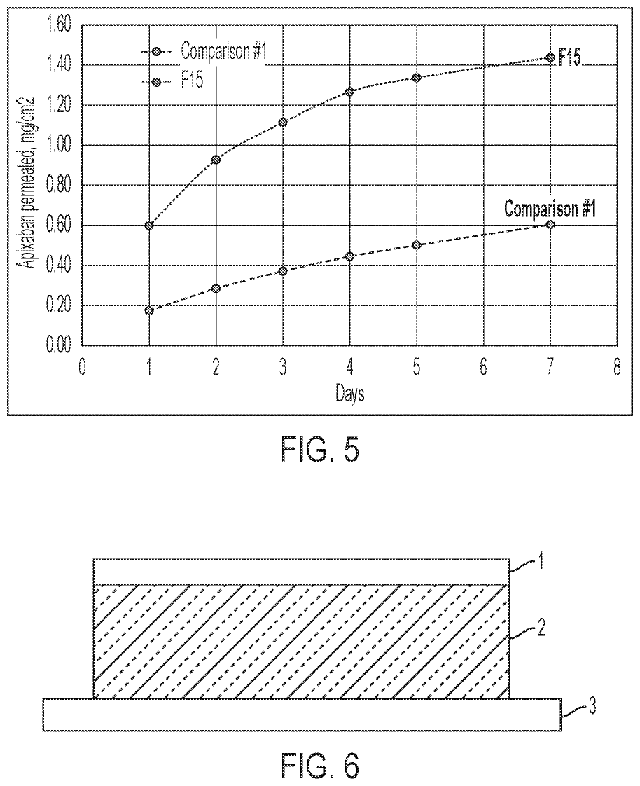

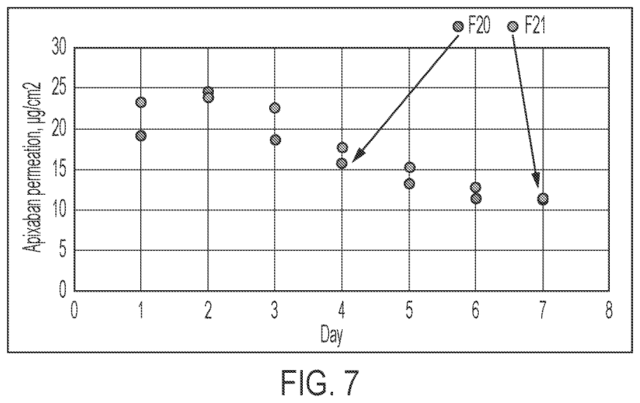

Skin permeation experiments were conducted side-by-side using the protocol of Example 2, but using a synthetic skin, PERMEAPAD, instead of human cadaver skin. The F15 gel is applied in a vast excess in the donor chamber layered on top of the skin membrane in the Franz cell. Thus, the results for the gel formulation are comparable to results obtained using a reservoir patch. The objective here compares the relative permeability of transdermal formulations of reservoir and drug-in-adhesive matrix patch. See, Table 4 and FIG. 5.

TABLE 5
Cumulative Apixaban Permeated (mg/cm²)

| Sample/Day | Comparison #1 | F15 |
|---|---|---|
| 1 | 0.174 | 0.599 |
| 2 | 0.285 | 0.927 |
| 3 | 0.371 | 1.112 |
| 4 | 0.445 | 1.266 |
| 5 | 0.501 | 1.336 |
| 7 | 0.604 | 1.438 |

FIG. 5 shows that the apixaban skin permeability of the drug-in-adhesive (DIA) formulation is not as good as the reservoir patch formulation due to “dilution” effect of the adhesive component is the formulation composition Nevertheless, DIA formulation provides adequate permeability.

### Example 5

### A. Patch Preparation

Two “drug-in-adhesive matrix” patch formulations were prepared. See, Table 6.

TABLE 6

Weight %

| Component | F20 | F21 |
|---|---|---|
| Pyruvic Acid | 49.8% | 35.9% |
| Oleic Acid | 2.7% | 9.2% |
| Levulinic Acid | 2.8% | 9.2% |
| BRIJ O20 | 0.6% | 0.6% |
| Apixaban | 6.5% | 6.7% |
| DUROTAK 87-2287 | 37.5% | 38.5% |
| | 100.0% | 100.0% |

A matrix transdermal patch shown in FIG. 6 was prepared. In FIG. 6, the backing film (1) is affixed atop the adhesive drug matrix (2), which is supported by an over-sized release liner film (3).

The matrix transdermal patch was prepared as follows by adding ethyl acetate to a 200-mL glass vial, followed by apixaban. The additional components noted in Table 6 were added and the blend was mixed to dissolve the apixaban. While mixing, the DUROTAK 87-2287 acrylic adhesive was added. The batch was mixed for 60 minutes or until it was homogeneous. The resulting wet solution was cast onto a release liner film (3M), using a casting applicator of 20 mils. The casting was dried in a forced-air oven at 80° C. for 10 minutes. After drying, the dried casting was laminated to a SCOTCHPAK 9723 (3M Drug Delivery Systems) patch backing film. The patch was die-cut into a 10, 50, or 100 cm² shape to provide various dosage requirements. The patches were pouched in multi-laminated aluminum pouches.

The resulting transdermal patch had an adhesive matrix thickness of 6 mils and contained 6.5% apixaban HPLC analysis confirmed that the patch contained about 6.5% of apixaban. The patch had good skin adhesion and adhered snugly on skin. The patch was die-cut to fix on the Franz cells for skin permeation study.

### B. Skin Permeation Study

Skin permeation experiments were conducted side-by-side using the protocol described in Example 2. Results are shown in Table 7 and reported as daily apixaban permeated (mg/cm²).

TABLE 7

μg/cm²/day

| Day/form | F20 | F21 |
|---|---|---|
| 1 | 19.10 | 23.31 |
| 2 | 24.61 | 23.96 |
| 3 | 18.69 | 22.65 |
| 4 | 15.70 | 17.78 |
| 5 | 13.28 | 15.30 |
| 6 | 11.49 | 12.85 |
| 7 | 11.32 | 11.40 |

### C. Stability Study

The stability of formulation F20 was evaluated. Specifically, pouched patches of formulation F20 were stored in environmental chambers of 25° C./60% RH and 40° C./75% RH, and periodically monitored for HPLC assay and color tests. See, Table 8.

TABLE 8

| | | Temperature/Relative Humidity | | | | | |
|---|---|---|---|---|---|---|---|
| | | 25° C./60% RH | | | 40° C./75% RH | | |
| Timepoints | t = 0 | 1 Mo | 3 Mo | 6 Mo | 1 Mo | 3 Mo | 6 Mo |
| Color | yellow | yellow | yellow | yellow | yellow | yellow | brown |
| %* | 99.6 | 95.9 | 93.5 | 92.1 | 90.9 | 88.4 | 87.1 |

The results show that the purity of apixaban after storage was about 99 to about 100% as measured by HPLC. The apixaban transdermal patch is considered stable when stored at (i) 25° C./60% RH up to 6 months of storage and (ii) at 40° C./75% RH up to 1 month of storage.

### Example 6: Comparison Studies

This example was performed using rivaroxaban (XARELTO) which is a direct oral anticoagulant of the Factor Xa inhibitor class.

Reservoir patches containing rivaroxaban were prepared. Table 9 includes the components of each formulation.

TABLE 9

Weight %

| Component | Formulation 1A | Formulation 1B |
|---|---|---|
| Rivaroxaban | 2.4 | 3.10 |
| DMSO | 12.4 | 12.6 |
| NMP | 7.2 | 7.2 |
| Pyruvic acid | 38.2 | 38.2 |
| Lactic Acid | 23.0 | 23.0 |
| Propylene Glycol | 5.3 | 5.3 |
| Oleic Acid | 5.3 | 5.3 |
| Levulinic Acid | 5.3 | 5.3 |
| Brij O-20 | 1.0 | 0 |
| Total | 100.0 | 100.0 |

Skin permeation studies using formulations 1A and 1B were performed as described in Example 1. See, Table 10 with the average drug permeated.

TABLE 10

Average drug permeated (μg/cm²/day)

| Day | Formulation 1A | Formulation 1B |
|---|---|---|
| 1 | 12.5 | 25.0 |
| 2 | 11.7 | 13.9 |
| 3 | 3.74 | 4.07 |
| 4 | 7.18 | 7.00 |
| 5 | 4.09 | 3.71 |
| 6 | 3.71 | 2.15 |
| 7 | 3.08 | 2.82 |

The results showed that transdermal delivery of apixaban was more effective than it was for rivaroxaban since the rivaroxaban formulations resulted in a very small amount of transdermal rivaroxaban permeation.

### Example 7: Summary

Examples 1-6 demonstrate that the transdermal delivery of apixaban, both in therapeutic quantities and in a steady, near-zero-order kinetic profile, is feasible with the appropriate selection of excipients and skin permeation enhancers.

Lower MW acids appear to improve skin peameability of apixaban.

### Example 8

This study will evaluate the pharmacokinetics of an apixaban formulation as described herein and ELIQUIS (apixaban tablets, for oral use) in healthy adult subjects 18 years of age or older.

The transdermal delivery of apixaban as described herein (rather than oral) will achieve approximate zero-order kinetics over the duration of wear. In general, the cumulative in vitro drug permeation through human cadaver skin at any given timepoint (μg/cm²) can be projected to represent the in vivo dose delivered to the circulation, since what crosses the skin is generally then 100% bioavailable to the systemic circulation, (i.e., no hepatic first-pass metabolism) through a 1 cm² patch. The data is analyzed over the first several days where the daily absorption is as close as possible to linear from day to day (i.e. the cumulative absorption curve is nearly a straight line), and use the cumulative drug permeation at the end of that number of days. The data is also analyzed over multiple timepoints on day 1 to determine whether the delivery is nearly linear (zero-order kinetics) at least for that day. Thus, the amount of apixaban delivered in vivo can be determined, by one of ordinary skill in the art, depending on the surface area of the transdermal patch.

### A. Detailed Description

Each subject is randomly assigned to one of the two groups according to a randomization table. In one group, subjects will receive ELIQUIS. In the other group, subjects will receive an apixaban formulation described herein.

### B. Eligibility Criteria

Inclusion and exclusion criteria will be:

### Inclusion Criteria:

1. Sign informed consent will be completed before the test, and subjects will fully understand the test content, process and possible adverse reactions;
2. Subjects will be able to complete the study according to the requirements of the test plan;
3. Subjects (including male subjects) will agree to have no pregnancy plan and to voluntarily take effective contraceptive measures within 3 months from the end of the study after signing the informed consent;
4. Male and female subjects will be 18 years and above;
5. Male subjects will weigh at least 50 kg. Female subjects weigh at least 45 kg. Body Mass index=weight (kg)/height 2 (m²), within the range of 18.0 to 26.0 kg/m² (including the critical value).

### Exclusion Criteria:

1. Prior disease of the neuropsychiatric, respiratory, cardiovascular, digestive, hematolymphatic, hepatic or renal insufficiency, endocrine, skeletal-muscular system, or other disease, and the investigator determines that this prior medical history may have an impact on drug metabolism or safety;
2. Persons who have smoked more than 5 cigarettes per day on average during the 3 months prior to screening;
3. Persons with a history of specific allergies (asthma, urticaria, eczema, etc.), allergic persons (e.g., allergy to two or more drugs, food), known hypersensitivity to this drug component or (including but not limited to) rivaroxaban;
4. Lactose intolerant individuals (e.g., those who have experienced diarrhea from drinking milk);
5. History of alcohol abuse (≥14 units of alcohol per week: 1 unit=285 ml of beer, or 25 ml of spirits over 40 degrees, or 100 ml of wine) within the last year;
6. Have donated blood or lost ≥400 ml of blood within 3 months prior to the trial, or intend to donate blood or blood components during or within 3 months after the trial;
7. A history of dysphagia or any gastrointestinal disorder that interferes with drug absorption;
8. Use of any recombinant cytochrome P450 3A4 (CYP3A4) or permeability glycoprotein (P-gp) inhibitors (e.g., ketoconazole, itraconazole, voriconazole and posaconazole, ritonavir, diltiazem, naproxen, amiodarone, verapamil, quinidine, famotidine, macrolides, nitroimidazoles, sedative-hypnotics, fluoroquinolones, antihistamines), CYP3A4 and P-gp inducers (e.g., rifampin, phenytoin, carbamazepine, phenobarbital or St. John's wort, omeprazole, glucocorticoids);
9. Any anticoagulant, platelet aggregation inhibitor, selective 5-hydroxytryptamine reuptake inhibitor or 5-hydroxytryptamine norepinephrine reuptake inhibitor or non-steroidal anti-inflammatory drug used within 30 days prior to the trial and drugs that may cause severe bleeding, such as common heparin and heparin derivatives (including low molecular weight heparin), oligosaccharides that inhibit coagulation factor Xa (e.g. sulforaphane sodium), prothrombin II direct inhibitors thrombolytic agents, thienopyridines (e.g., clopidogrel), dipyridamole, dextran, sulpiride, vitamin K antagonists, and other oral anticoagulants;
10. Taking any prescription, over-the-counter, herbal or nutraceutical medication within 14 days prior to the administration of the study drug;
11. Have taken a special diet (caffeine, alcohol, or xanthine-rich foods and beverages, including dragon fruit, mango, grapefruit, chocolate, or tea), or smoked, or had a significant change in exercise habits, or other factors affecting drug absorption, distribution, metabolism, or excretion within 48 h prior to the administration of the study drug;
12. Those with a positive alcohol screening test;
13. Those who have participated in another clinical trial of a drug and taken the test drug within 3 months prior to taking the study drug;
14. Abnormalities of clinical significance as judged by the clinician, including physical examination, vital signs examination, electrocardiogram examination or clinical laboratory examination (including routine blood, urine, blood biochemistry, coagulation function examination);
15. Those with creatinine clearance≤50 mL/min;
16. Those with coagulation disorders, or those with bleeding tendencies (e.g., frequent recurrent gum bleeding), or those with increased bleeding risk events within the past 6 months, previous intracranial bleeding, gastrointestinal bleeding, purpura, or those who have undergone major surgery within the past 30 days, or those with active pathologic bleeding;
17. Positive for hepatitis B surface antigen, positive for hepatitis C antibodies, positive for HIV antibodies, or positive for primary syphilis screening;
18. Those with positive substance abuse screening or a history of substance abuse within the past five years;
19. Subjects with a history of blood or needle sickness, difficulty in blood collection or inability to tolerate venipuncture blood collection;
20. Subjects who are unable or incapable of complying with ward management regulations:
21. Subjects who have had a major illness or major surgical procedure within 4 weeks prior to administration of study drug;
22. Subjects who are unable to complete the trial for personal reasons;
23. Subjects who are judged by the investigator to be unfit to participate in the study of this trial;
24. Female subjects who are breastfeeding or have a positive pregnancy test result during the screening period or during the course of the trial;
25. Female subjects who are using oral contraceptives 30 days prior to taking the study drug and during the trial;
26. Female subjects using long-acting estrogen or progestin injections or implant tablets within 6 months prior to study drug administration and during the trial;
27. Female subjects who had unprotected sex two weeks prior to screening.

### C. Primary Outcome Measures

The following outcome measures will be determined and compared with those obtained for ELIQUIS:
Peak Plasma Concentration (Cₘₐₓ)
Area under the plasma concentration versus time curve (AUC₀₋ₜ)
Area under the plasma concentration versus time curve (AUC_{0-∞})
Safety and tolerability as determined by adverse event reporting, clinical laboratory results, vital signs, physical examinations, and ECGs.

The disclosures of each patent, patent application, and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A pharmaceutical composition comprising apixaban and pyruvic acid.

2. The pharmaceutical composition of claim 1, further comprising a surfactant, permeation enhancer, or a combination thereof.

3. The pharmaceutical composition of claim 2, wherein the surfactant or permeation enhancer is dimethylsulfoxide, lactic acid, oleic acid, levulinic acid, 3-hydroxypropionic acid, malonic acid, or a nonionic surfactant such as a polysorbate such as TWEEN 80, or a polyethylene glycol monooleyl ether, such as BRIJ O20.

4. The pharmaceutical composition of any one of the preceding claims, further comprising lactic acid.

5. The pharmaceutical composition of claim 3 or 4, comprising about 20 to about 45% by weight, based on the weight of the pharmaceutical composition, of lactic acid, or such as about 25 to about 35% by weight.

6. The pharmaceutical composition of any one of the preceding claims, further comprising dimethylsulfoxide.

7. The pharmaceutical composition of claim 6, comprising about 5 to about 20% by weight, based on the weight of the pharmaceutical composition, of dimethylsulfoxide, or about 8 to about 15% by weight.

8. The pharmaceutical composition of any one of the preceding claims, further comprising levulinic acid.

9. The pharmaceutical composition of claim 8, comprising about 1 to about 20% by weight, based on the weight of the pharmaceutical composition, of levulinic acid, ore such as about 3 to about 10% by weight.

10. The pharmaceutical composition of any one of the preceding claims, further comprising oleic acid.

11. The pharmaceutical composition of claim 10, comprising about 1 to about 10% by weight, based on the weight of the pharmaceutical composition, of oleic acid, or such as about 3 to about 7% by weight.

12. The pharmaceutical composition of any one of the preceding claims, further comprising a polysorbate, or such as polysorbate 80.

13. The pharmaceutical composition of claim 12, comprising about 5 to about 15% by weight, based on the weight of the pharmaceutical composition, of the polysorbate, or such as about 7 to about 12% by weight.

14. The pharmaceutical composition of any one of the preceding claims, further comprising a polyethylene glycol monooleyl ether.

15. The pharmaceutical composition of claim 14, comprising about 0.01 to about 5% by weight, based on the weight of the pharmaceutical composition, of the polyethylene glycol monooleyl ether, or such as about 0.5 to about 2% by weight.

16. The pharmaceutical composition of any one of the preceding claims, comprising about 5 to about 25% by weight, based on the weight of the pharmaceutical composition, of the anticoagulant, or such as about 7 to about 12% by weight, such as about 9%, about 10%, or about 11% by weight.

17. The pharmaceutical composition of any one of the preceding claims, comprising about 20 to about 90% by weight, based on the weight of the pharmaceutical composition, of pyruvic acid, or such as about 25 to about 45% by weight, or about 30 to about 45% by weight.

18. The pharmaceutical composition of any one of the preceding claims, comprising about 0.1 to about 100 mg/day, or such as about 2.5 mg/day, about 5 mg/day, about 10 mg/day, about 15 mg/day, or about 20 mg of apixaban.

19. The pharmaceutical composition of any one of the preceding claims that lacks acrylic acid.

20. A dosage form comprising the pharmaceutical composition of any one of the preceding claims.

21. The dosage form of claim 20 that is a transdermal patch.

22. The dosage form of claim 21, wherein the transdermal patch is a single-layer drug-in-adhesive patch, multi-layer drug-in-adhesive patch, reservoir patch, matrix patch, or a vapor patch, or such as a reservoir patch.

23. The dosage form of any one of claims 20-22, further comprising a pressure sensitive adhesive.

24. A method for treating a thromboembolic disorder in a patient in need thereof, comprising administering the pharmaceutical composition of any one of claims 1-19 or the dosage form of any one of claims 20-22 to the patient.

25. The method of claim 24, wherein the thromboembolic disorder is an arterial cardiovascular thromboembolic disorder, venous cardiovascular thromboembolic disorder, or thromboembolic disorder in the chambers of the heart.

26. The method of claim 24, wherein the thromboembolic disorder is an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or a thrombosis resulting from or a procedure in which blood is exposed to an artificial surface that promotes thrombosis such as thrombosis resulting from an implant such as a prosthetic valve, thrombosis resulting from an indwelling catheter, thrombosis resulting from a stent, thrombosis resulting from a cardiopulmonary bypass, thrombosis resulting from hemodialysis, thrombosis resulting from or a procedure in which blood is exposed to an artificial surface that promotes thrombosis.

27. The method of claim 24, wherein the thromboembolic disorder is an acute coronary syndrome.

28. The method of claim 24, wherein the thromboembolic disorder is stroke.

29. The method of claim 24, wherein the thromboembolic disorder is deep vein thrombosis.

30. The method of claim 24, wherein the thromboembolic disorder is pulmonary embolism.

31. A method for preventing a deep vein thrombosis or a pulmonary embolism in a patient in need thereof, comprising administering the pharmaceutical composition of any one of claims 1-19 or the dosage form of any one of claims 20-23 to the patient.

32. A method for reducing the reoccurrence of a deep vein thrombosis or a pulmonary embolism in a patient in need thereof, comprising administering the pharmaceutical composition of any one of claims 1-19 or the dosage form of any one of claims 20-23 to the patient.

33. The method of claim 31 or 32, wherein the patient had hip or knee replacement surgery.

34. A method for preventing a venous thromboembolism (VTE) in an acutely ill patient, comprising administering the pharmaceutical composition of any one of claims 1-19 or the dosage form of any one of claims 20-23 to the patient.

35. A method for reducing the risk of a major cardiovascular event in a patient with coronary artery disease, comprising administering the pharmaceutical composition of any one of claims 1-19 or the dosage form of any one of claims 20-23 to the patient.

36. A method for reducing the risk of a major thrombotic vascular event in a patient with peripheral artery disease (PAD), comprising administering the pharmaceutical composition of any one of claims 1-19 or the dosage form of any one of claims 20-23 to the patient.

37. The method of claim 36, wherein the patient had recent lower extremity revascularization due to symptomatic PAD.

38. A method for treating VTE or reducing the risk of reoccurrence of VTE in a pediatric patient in need thereof, comprising administering the pharmaceutical composition of any one of claims 1-19 or the dosage form of any one of claims 20-23 to the patient.

39. A method for thromboprophylaxis in a patient with congenital heart disease after a Fontan procedure, comprising administering the pharmaceutical composition of any one of claims 1-19 or the dosage form of any one of claims 20-23 to the patient.

40. The method of claim 38, wherein the patient is 2 years of age or older.

41. A method for treating atherosclerosis, myocardial infarct, pulmonary embolism or deep venous thrombosis in a patient in need thereof, comprising administering the pharmaceutical composition of any one of claims 1-19 or the dosage form of any one of claims 20-23 to the patient.

42. The method of any one of claims 24-41, wherein the pharmaceutical composition is administered transdermally.

43. The method of any one of claims 24-42, further comprising administering a second anticoagulant.

44. The method of claim 43, wherein the second anticoagulant is aspirin.

45. The method of any one of claim 31-37 or 39-44, wherein the patient is an adult.

46. The method of any one of claims 31-45, wherein the patient is a child.

47. A method of preventing one or more thromboembolic or hemorrhagic events in a patient with atrial fibrillation, comprising administering the pharmaceutical composition of any one of claims 1-19 or the dosage form of any one of claims 20-23 to the patient.

48. A method for reducing the risk of reducing the risk of stroke or systemic embolism in a patient, comprising administering the pharmaceutical composition of any one of claims 1-19 or dosage form of any one of claims 20-23 to the patient.

49. The method of claim 48, wherein the patient has nonvalvular atrial fibrillation.
